(19) United States
(12) Patent Application Publication
(10) Pub. No.: US 2026/0258222 A1
(43) Pub. Date: Sep. 3, 2026

(54) COMPOUND, CURABLE RESIN COMPOSITION AND CURED PRODUCT THEREOF

(71) Applicant: NIPPON KAYAKU KABUSHIKI KAISHA, Tokyo (JP)

(72) Inventors: Takayuki TOHJIMA, Tokyo (JP); Kenichi KUBOKI, Tokyo (JP); Masanori HASHIMOTO, Tokyo (JP); Masataka NAKANISHI, Tokyo (JP)

(73) Assignee: NIPPON KAYAKU KABUSHIKI KAISHA, Tokyo (JP)

(21) Appl. No.: 18/995,882

(22) PCT Filed: Nov. 24, 2023
(86) PCT No.: PCT/JP2023/042116

§ 371 Date: Jan. 17, 2025

(30) Foreign Application Priority Data
Nov. 25, 2022 (JP) 2022-187894

(51) Int. Cl. C08K 5/3415; C07D 207/412; C08G 73/10

(52) U.S. Cl. CPC C08K 5/3415; C07D 207/412; C08G 73/10

(57) ABSTRACT

The present invention provides a compound having excellent heat resistance and low dielectric properties, and a curable resin composition and a cured product thereof. The compound is obtained by reacting a compound represented by formula (a) with a compound having two or more amino groups per molecule. (In formula (a), Y represents a C1-20 hydrocarbon group.)

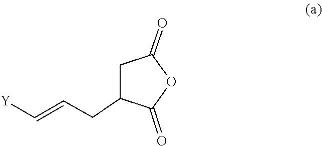

### TECHNICAL FIELD

The present invention relates to a compound having a specific structure, a curable resin composition, and a cured product thereof, which are suitably used for electrical and electronic components such as semiconductor encapsulants, printed circuit boards, and build-up laminates, lightweight high-strength materials such as carbon fiber reinforced plastics and glass fiber reinforced plastics, and 3D printing applications.

### RELATED ART

In recent years, the required properties for laminates used for mounting electrical and electronic components have become more diverse and sophisticated due to the expansion of their application fields. While conventional semiconductor chips are primarily mounted on metal lead frames, high-performance semiconductor chips such as central processing units (hereinafter referred to as CPU) are increasingly being mounted on laminates made of polymeric materials.

Especially in terms of semiconductor packages (hereinafter referred to as PKG) used in smartphones or the like, there is a demand for thinner PKG substrates to meet the requirements of miniaturization, thinning, and high density. However, as PKG substrates become thinner, the rigidity decreases, which leads to issues such as significant warpage that occurs due to heating when solder mounting PKG onto the motherboard (PCB). To mitigate this, PKG substrate materials with high Tg above the solder mounting temperature are required.

Additionally, in the fifth-generation communication system “5G”, which is currently undergoing accelerated development, further increases in capacity and communication speed are anticipated. As 5G uses increasingly higher frequencies, reducing transmission loss becomes crucial for achieving high-speed communication using high frequencies, and substrate materials with even lower dielectric properties are required. Transmission loss occurring on printed circuit boards originates from conductor loss and dielectric loss. As described in Non-Patent Literature 1, conductor loss is proportional to the square root of the relative dielectric constant and the dielectric loss tangent of the dielectric. Therefore, it can be said that improving the dielectric loss tangent, which has a higher contribution than the relative dielectric constant, is more effective in reducing transmission loss. Thermoplastic materials such as PTFE (polytetrafluoroethylene) and LCP (liquid crystal polymer) are known as low dielectric materials, but thermoplastic materials have poor formability compared to thermosetting resins. In light of this, it is desired to develop of thermosetting resins with excellent low dielectric properties.

Furthermore, in the automotive field, as electronics become more prevalent, precision electronic devices may be placed near engine drive parts and therefore require higher levels of heat and moisture resistance. SiC semiconductors are beginning to be used in trains and air conditioners, and the encapsulants for semiconductor elements require extremely high heat resistance. As a result, conventional epoxy resin encapsulants can no longer meet these requirements.

In response to this background, bismaleimide compounds have recently been considered to achieve both heat resistance and electrical properties superior to epoxy resins. However, in the case of general-purpose bismaleimide compounds, they have drawbacks such as poor solvent solubility and can only be dissolved in high-boiling point non-protic polar solvents. Although it is possible to impart solvent solubility by introducing special molecular structures as in Patent Literature 1, there are also limitations in the types of amine compounds that can be used as the raw materials. Therefore, a more straightforward method to impart solvent solubility is desired.

In addition, Patent Literatures 2 and 3 have investigated polymeric materials that can achieve both heat resistance and low dielectric loss tangent properties. For example, Patent Literature 2 proposes a composition including a maleimide compound and a propenyl group-containing phenolic resin. However, on the other hand, phenolic hydroxyl groups that do not participate in the reaction during curing reaction remain, so the electrical properties cannot be considered sufficient. Also, Patent Literature 3 discloses an allyl ether resin in which hydroxyl groups are substituted with allyl groups. However, it is shown that Claisen rearrangement occurs at 190° C., and at 200° C., which is the general molding temperature for substrates, phenolic hydroxyl groups that do not contribute to the curing reaction are generated, so it cannot satisfy the electrical properties.

### CITATION LIST

### Non-Patent Literature

Non-Patent Literature 1: Signal Loss Factors in High-Speed Signal Transmission on Printed Circuit Boards (Mitsui Mining and Smelting Co., Ltd.) The 29th Spring Conference of the Japan Institute of Electronics Packaging 16P1-17

### PATENT LITERATURES

Patent Literature 1: Japanese Patent Application Laid-Open No. 2018-012671
Patent Literature 2: Japanese Patent Application Laid-Open No. H04-359911
Patent Literature 3: International Publication No. 2016/002704

### SUMMARY OF INVENTION

### Technical Problem

The present invention, in view of such circumstances, aims to provide a compound having excellent heat resistance and low dielectric properties, as well as a curable resin composition and a cured product thereof.

### Solution to Problem

That is, the present invention relates to [1] to [7] below. In this application, “(numerical value 1) to (numerical value 2)” indicates that the upper and lower limit values are included.

[1]

A compound obtained by reacting a compound represented by the following formula (a) with a compound having two or more amino groups in a molecule.

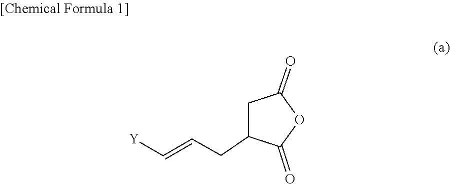

(In the above formula (a), Y represents a hydrocarbon group having 1 to 20 carbon atoms.)

[2]

The compound according to [1], wherein the compound having two or more amino groups in the molecule has an alicyclic ring or an aromatic ring.

[3]

The compound according to [1] or [2], represented by the following formula (b).

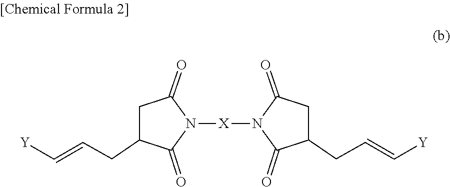

(In the above formula (b), multiple Y independently represent hydrocarbon groups having 1 to 20 carbon atoms, and X represents one or more of the following formulas (A) to (T).)

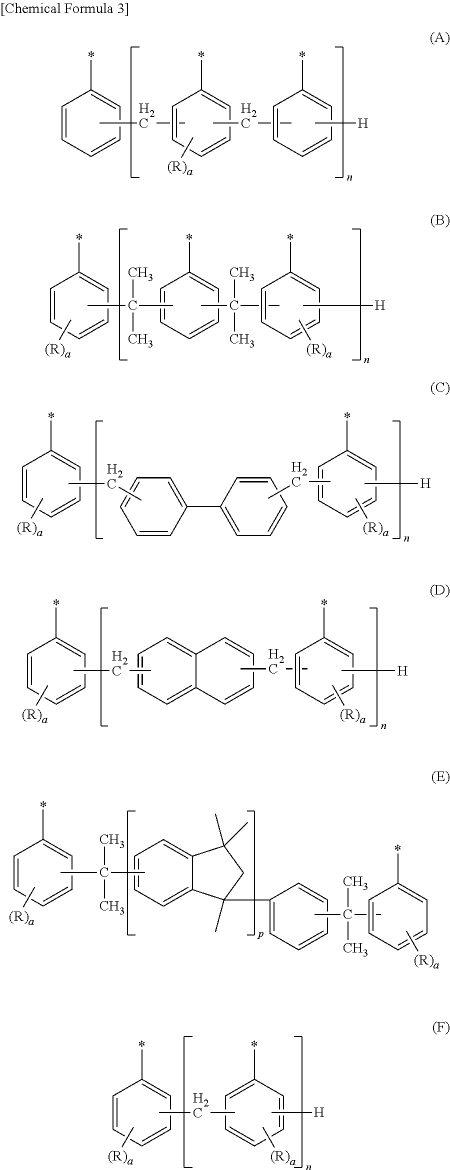

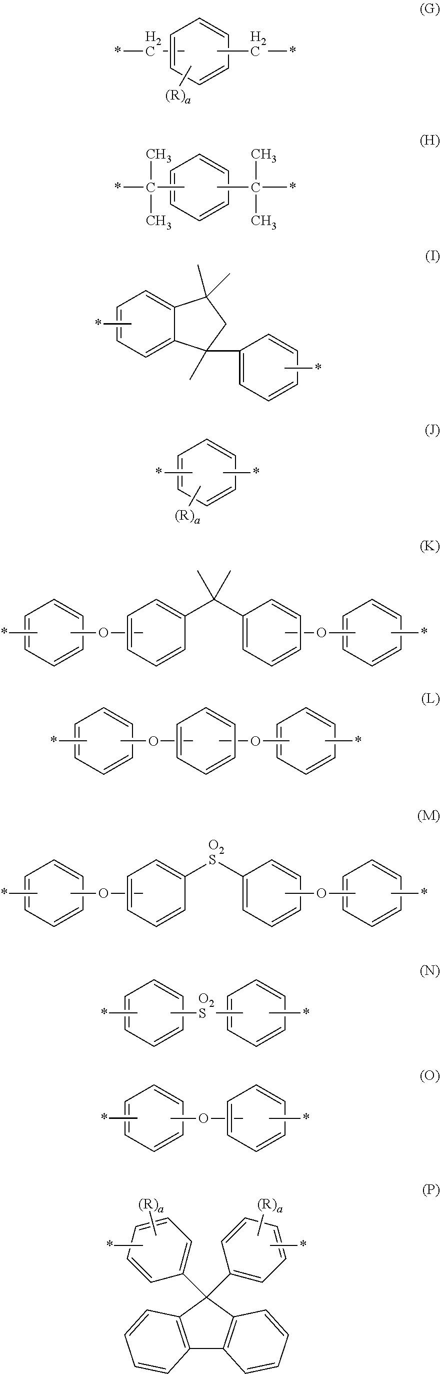

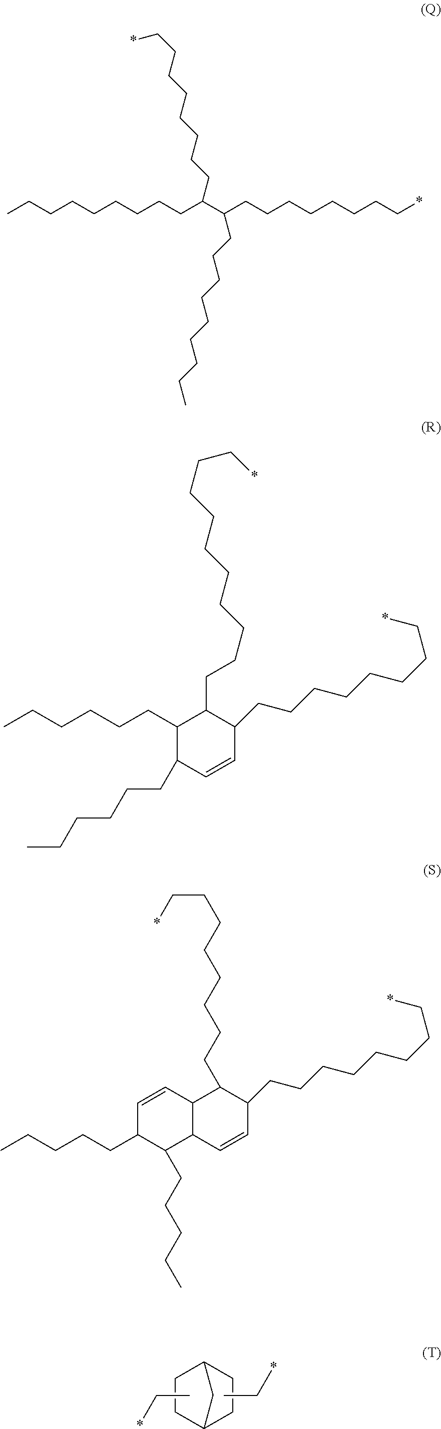

(In the above formulas (A) to (T), R represents a hydrogen atom or a hydrocarbon group having 1 to 10 carbon atoms, and a represents an integer of 1 to 4. p and n are each the number of repetitions and represent a number of 1 to 20. * indicates a bonding position with a nitrogen atom in formula (2).)

[4] A curable resin composition, including the compound according to any one of [1] to [3].
[5]

The curable resin composition according to [4], further including a polymerization initiator. [6]

The curable resin composition according to [4] or [5], further including a maleimide compound.

[7]

A cured product, obtained by curing the curable resin composition according to any one of [4] to [6].

### Effects of Invention

The compound and the curable resin composition of the present invention are excellent in high heat resistance and low dielectric properties.

### BRIEF DESCRIPTION OF DRAWINGS

FIG. 1 shows a GPC chart of Example 1.

FIG. 2 shows a GPC chart of Example 2.

### DESCRIPTION OF EMBODIMENTS

The compound of the present invention is obtained by reacting a compound represented by the following formula (a) with a compound having two or more amino groups in the molecule.

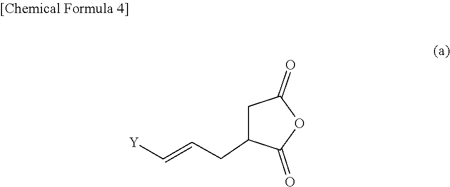

In the above formula (a), Y represents a hydrocarbon group having 1 to 20 carbon atoms, more preferably 2 to 20 carbon atoms, and even more preferably 4 to 20 carbon atoms. When the number of carbon atoms is 1 or more, the solvent solubility becomes favorable and the water absorption rate also decreases. Moreover, when the number of carbon atoms is 20 or less, the heat resistance and solvent solubility also become favorable, and purification by water washing becomes possible. As specific examples of the compound represented by the aforementioned formula (a), specific examples of the compound represented by the aforementioned formula (a) include tetrapropenylsuccinic anhydride (common name TPSA, a compound where Y in formula (a) is C₉H₁₉; the compound may include a mixture of isomers), dodecenylsuccinic anhydride (common name n-DDSA, a compound where Y in formula (a) is C₉H₁₉), octenylsuccinic anhydride (a compound where Y in formula (a) is C₅H₁₁), nonenylsuccinic anhydride (common name NSA, a compound where Y in formula (a) is C₆H₁₃), octadecenylsuccinic anhydride (Y in formula (a) is C₁₅H₃₁; the compound may include a mixture of isomers), tetradecenylsuccinic anhydride (a compound where Y in formula (a) is C₁₁H₂₃), etc., but not limited to these. The compound represented by the aforementioned formula (a) may be used alone, or compounds with different Y may be used in combination.

Any known amine compound may be used as the compound having two or more amino groups in the molecule. Examples include the amine compounds represented by the following formulas (A′) to (T′), methylenediamine, ethylenediamine, propylenediamine, butylenediamine, pentamethylenediamine, hexamethylenediamine, trimethylhexamethylenediamine, dimer diamine, 1,3-bis(aminomethyl)cyclohexane, isophoronediamine, bis(aminomethyl)tricyclodecane, dimer diamine, etc. It is preferable to use one or more of the following formulas (A′) to (T′), and it is even more preferable to use one or more of the following formulas (A′) to (G′), and (R′) to (T′). Moreover, it is possible to improve heat resistance and solvent solubility by using an aromatic amine compound or an alicyclic amine compound having molecular weight distribution. These may be used alone or in combination. It should be noted that dimer diamine refers to diamine in which the two terminal carboxyl groups (—COOH) of dimer acid are substituted with primary aminomethyl groups (—CH₂—NH₂) or amino groups (—NH₂). Known commercially available products may be used as the dimer diamine. Examples of the commercially available products include Priamine (registered trademark) manufactured by Croda Japan, etc.

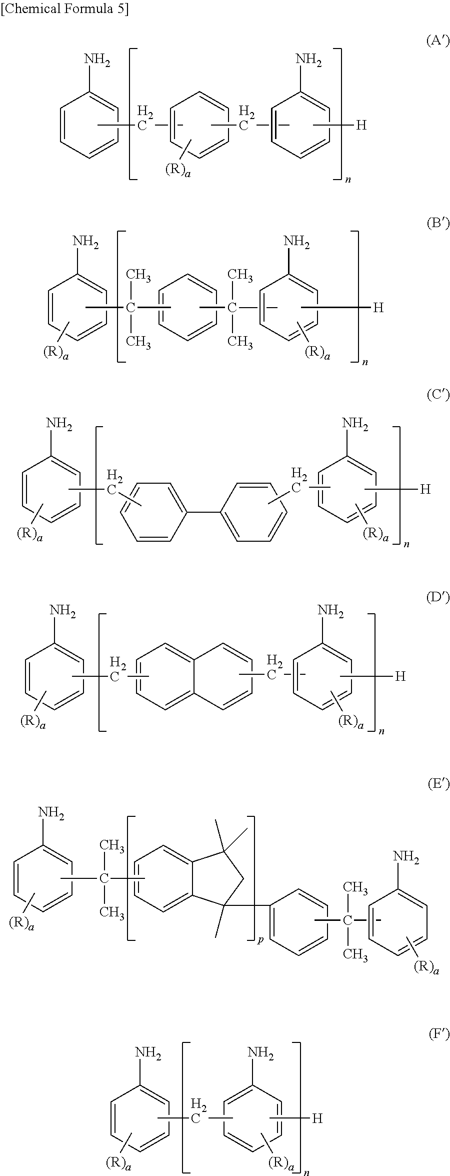

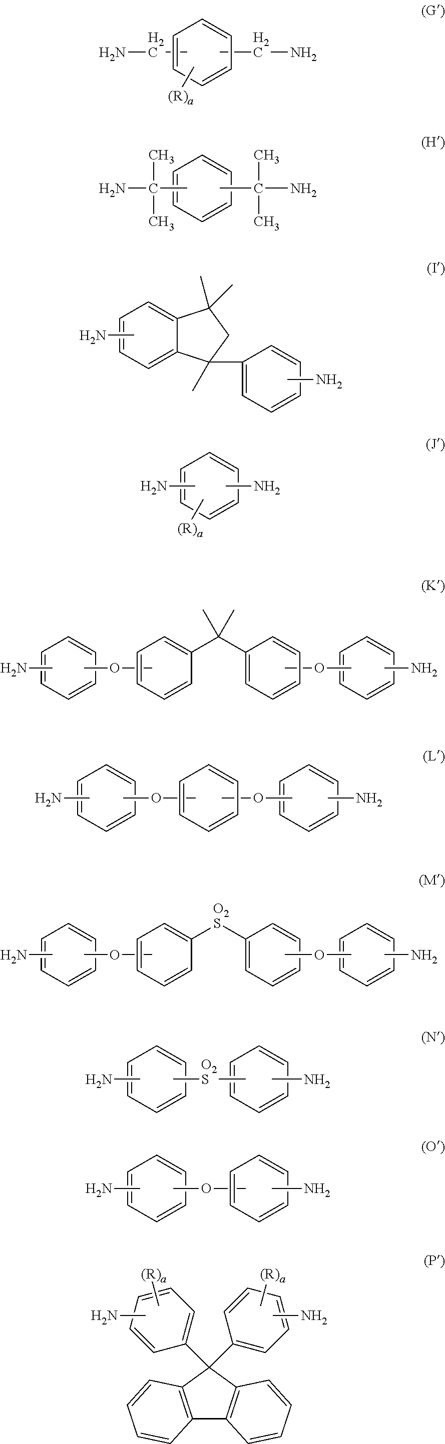

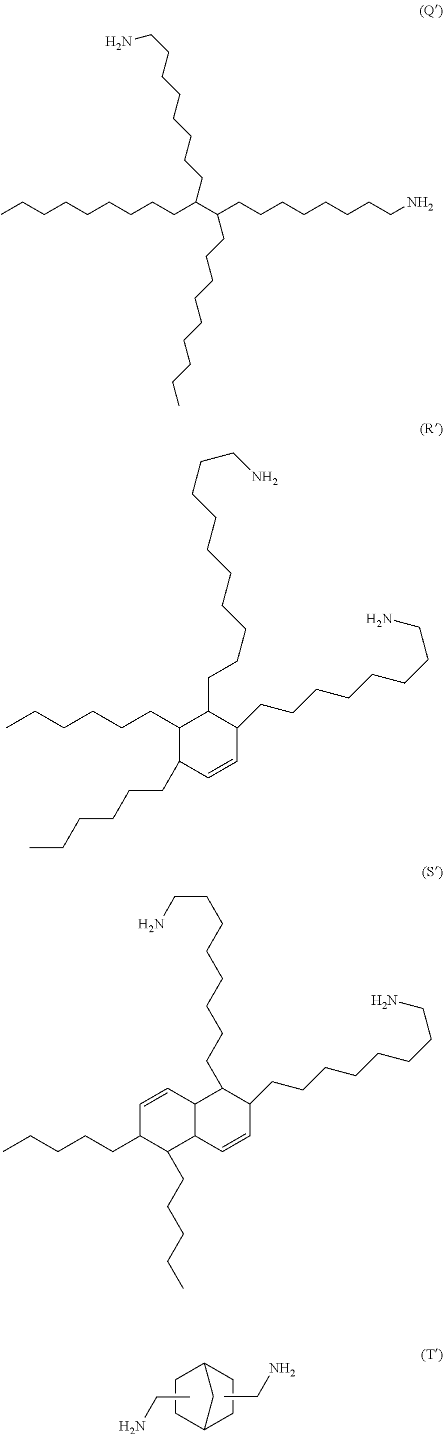

In the above formulas (A′) to (T′), R represents a hydrogen atom or a hydrocarbon group having 1 to 10 carbon atoms, and a represents an integer of 1 to 4. p and n are each the number of repetitions and represent an integer of 1 to 20.

It should be noted that the average value p of the number of repetitions in formula (E′), and the average value n of the number of repetitions in formulas (A′) to (D′) and (F′) can be calculated from the number average molecular weight (Mn) value obtained by gel permeation chromatography (hereinafter also referred to as GPC) measurement of the compounds of formulas (A′) to (E′), or from the area % of slice data of each peak (detector: differential refractive index detector), etc.

In the above formulas (A′) to (T′), R is typically a hydrocarbon group having 1 to 10 carbon atoms, preferably 1 to 5 carbon atoms, and more preferably 1 to 3 carbon atoms. When R is a hydrocarbon having 10 or fewer carbon atoms, it is less likely to undergo molecular vibration in response to exposure to high frequencies, thus resulting in excellent electrical properties. Moreover, it is preferable when n is in the range of 1<n<5.

In the above formula (B′), it is particularly preferable when a is 0, that is, when the compound is represented by the following formula (1).

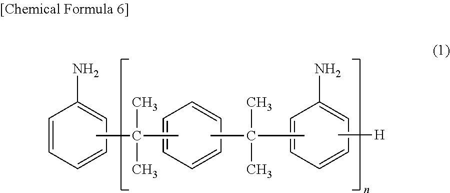

In formula (1), n is the average value of the number of repetitions, where 1≤n<5. The average value n of the number of repetitions in formula (1) can be calculated from the number average molecular weight (Mn) value obtained by gel permeation chromatography (hereinafter also referred to as GPC) measurement of the compound of formula (1), or from the area % of slice data of each peak (detector: differential refractive index detector), etc.

The above formula (E′) is particularly preferable when the compound is represented by the following formula (2).

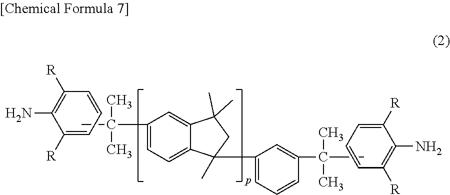

In formula (2), each R exists independently and represents a hydrocarbon group having 1 to 3 carbon atoms, and p is the average value of the number of repetitions, where 0≤p≤20. The average value p of the number of repetitions in formula (2) can be calculated from the number average molecular weight (Mn) value obtained by gel permeation chromatography (hereinafter also referred to as GPC) measurement of the compound of formula (2), or from the area % of slice data of each peak (detector: differential refractive index detector), etc.

The above formula (F′) is particularly preferable when a is 2 and n is 0, that is, when the compound is represented by the following formula (3), or when a is 0, that is, when the compound is represented by the following formula (4).

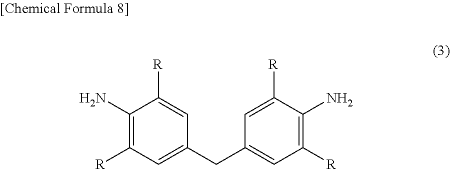

In formula (3), each R exists independently and represents a hydrocarbon group having 1 to 3 carbon atoms.

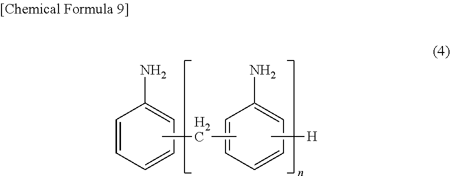

In formula (4), n is the average value of the number of repetitions, where 1≤n<5. The average value n of the number of repetitions in formula (4) can be calculated from the number average molecular weight (Mn) value obtained by gel permeation chromatography (hereinafter also referred to as GPC) measurement of the compound of formula (4), or from the area % of slice data of each peak (detector: differential refractive index detector), etc.

The above formula (C′) is particularly preferable when a is 0, that is, when the compound is represented by the following formula (5).

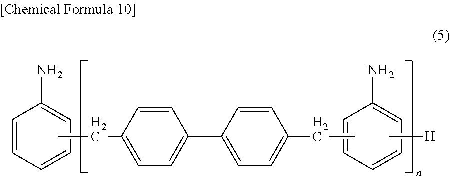

In formula (5), n is the average value of the number of repetitions, where 1≤n<5. The average value n of the number of repetitions in formula (5) can be calculated from the number average molecular weight (Mn) value obtained by gel permeation chromatography (hereinafter also referred to as GPC) measurement of the compound of formula (5), or from the area % of slice data of each peak (detector: differential refractive index detector), etc.

The compound of the present invention is obtained by reacting a compound represented by the aforementioned formula (a) with a compound having two or more amino groups in the molecule, and can be represented by the following formula (b).

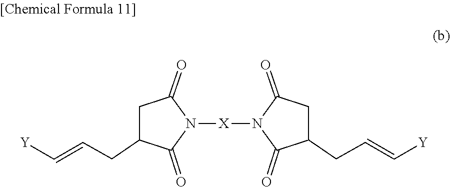

In the above formula (b), multiple Y each independently represent a hydrocarbon group having 1 to 20 carbon atoms, preferably 2 to 20 carbon atoms, and more preferably 4 to 20 carbon atoms. When the number of carbon atoms is 1 or more, the solvent solubility becomes favorable and the water absorption rate decreases. Additionally, when the number of carbon atoms is 20 or less, the heat resistance and solvent solubility also become favorable, and purification by water washing becomes possible. Since Y is derived from the aforementioned formula (a), specific examples include C₉H₁₉, C₅H₁₁, C₆H₁₃, C₁₅H₃₁, C₁₁H₂₃, etc., but not limited to these. The compound represented by the aforementioned formula (a) may be used alone, or compounds with different Y may be used in combination.

In the above formula (b), X is derived from the compound having two or more amino groups in the molecule, and therefore can take a structure derived from the compound having two or more amino groups in the molecule. However, it is preferable when X is one or more of the following formulas (A) to (T), and it is more preferable when X is one or more of the following formulas (A) to (G), and (R) to (T).

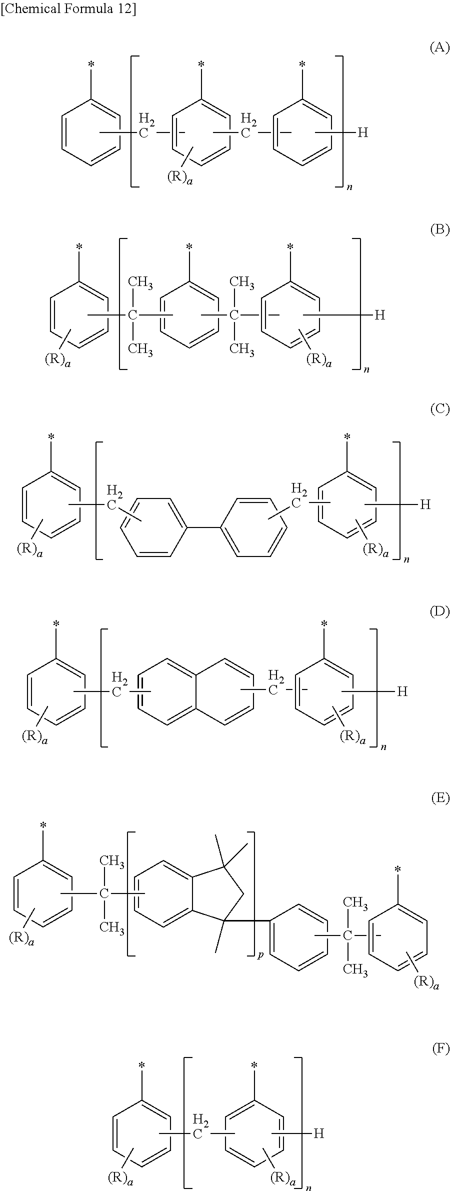

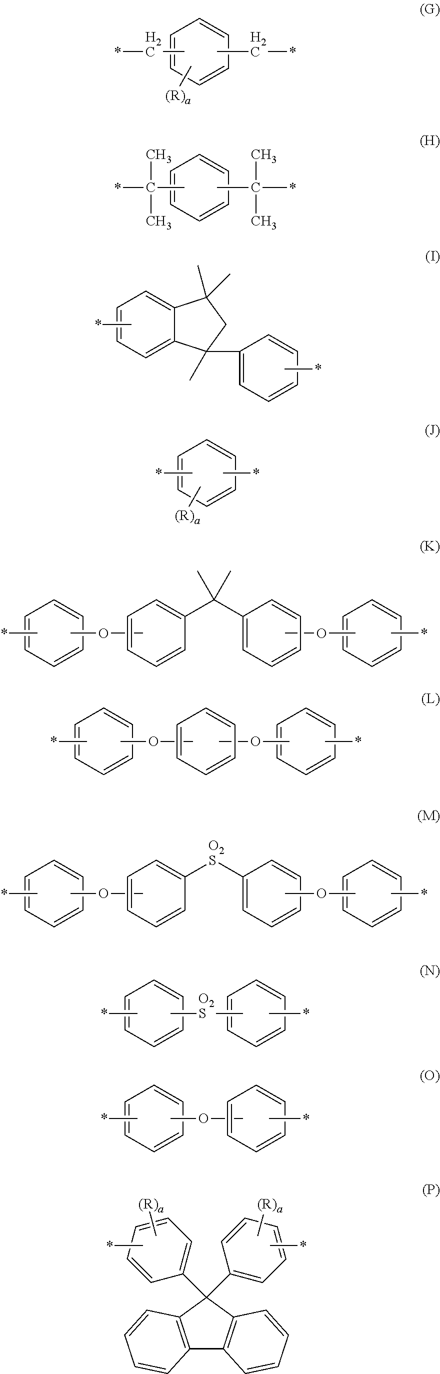

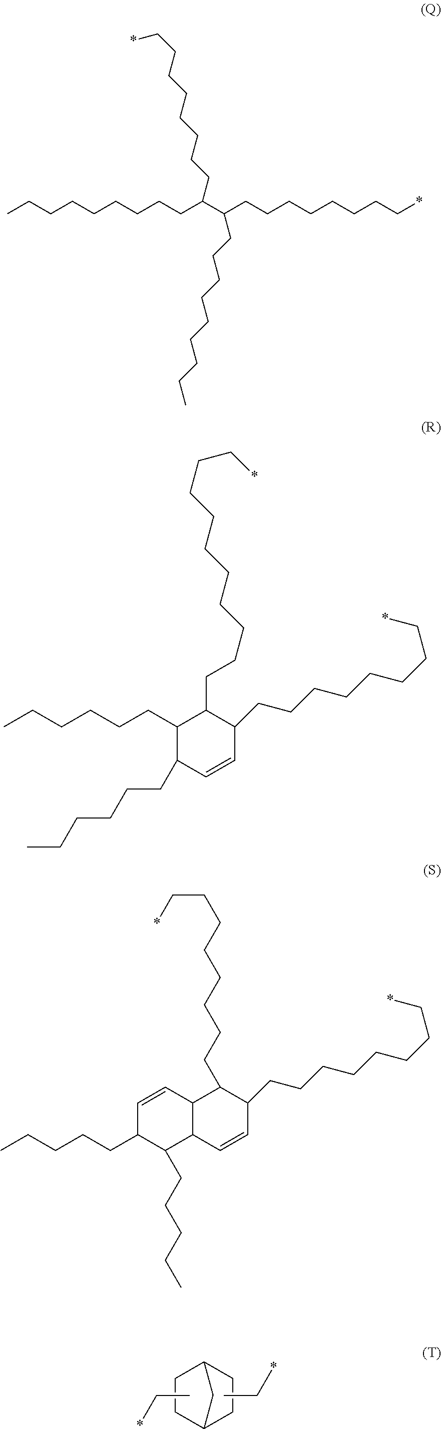

In the above formulas (A) to (T), R represents a hydrogen atom or a hydrocarbon group having 1 to 10 carbon atoms, and a represents an integer of 1 to 4. p and n are each the number of repetitions and represent a number of 1 to 20. * indicates the bonding position with the nitrogen atom in formula (2).

In the above formulas (A) to (T), R is typically a hydrocarbon group having 1 to 10 carbon atoms, preferably a hydrocarbon group having 1 to 5 carbon atoms, and more preferably a hydrocarbon group having 1 to 3 carbon atoms. When R is a hydrocarbon having 10 or fewer carbon atoms, it is less likely to undergo molecular vibration in response to high frequencies, resulting in excellent electrical properties.

The method of manufacturing the compound of the present invention is not particularly limited, but the compound can be derived from a compound represented by the aforementioned formula (a) and a compound having two or more amino groups in the molecule.

Specifically, the compound can be obtained by a method of reacting and cyclizing a compound represented by the aforementioned formula (a) with a compound having two or more amino groups in the molecule in the presence of a catalyst, in a solvent, at a temperature range of 0° C. to 200° C., preferably 0° C. to 150° C., to achieve imidization. Examples of the solvent to be used include non-aqueous solvents such as aromatic solvents like toluene and xylene, aliphatic solvents like cyclohexane and n-hexane, ethers like diethyl ether and diisopropyl ether, ester solvents like ethyl acetate and butyl acetate, ketone solvents like methyl isobutyl ketone and cyclopentanone, but not limited to these, and two or more may be used in combination. In addition to the aforementioned non-aqueous solvents, aprotic polar solvents can also be used in combination. Examples include dimethyl sulfone, dimethyl sulfoxide, dimethylformamide, dimethylacetamide, 1,3-dimethyl-2-imidazolidinone, N-methylpyrrolidone, etc., and two or more may be used in combination. In the case of using aprotic polar solvents, it is preferable to use an aprotic polar solvent with a higher boiling point than the non-aqueous solvent used in combination. During the reaction, if necessary, catalysts such as hydrochloric acid, phosphoric acid, sulfuric acid, formic acid, p-toluenesulfonic acid, and methanesulfonic acid, as well as Lewis acids such as aluminum chloride and zinc chloride, solid acids such as activated clay, acid clay, white carbon, zeolite, silica alumina, acidic ion exchange resins, etc. can be used. These may be used alone or in combination of two or more. The usage amount of the catalyst is typically 0.1 moles to 0.8 moles, preferably 0.2 moles to 0.7 moles, per mole of amino group of the amine compound used. When the usage amount of the catalyst is too high, there is a risk that the viscosity of the reaction solution becomes too high, making stirring difficult; and when the usage amount is too low, there is a risk that the reaction progress becomes slow. Also, basic co-catalysts such as triethylamine can be used alone or in combination as co-catalysts for imidization. In the case of using sulfonic acid, etc. as the catalyst, neutralization may be performed with alkali metals such as sodium hydroxide and potassium hydroxide before proceeding to the extraction process. For the extraction process, aromatic hydrocarbon solvents such as toluene and xylene may be used alone, or non-aromatic hydrocarbons such as cyclohexane and toluene may be used in combination.

After extraction, the organic layer is washed with water until the wastewater becomes neutral, and the solvent is distilled off using an evaporator, etc. to obtain the desired compound.

### [Curing Accelerator]

The curable resin composition of the present invention can also improve curability by adding a curing accelerator. As the curing accelerator, an anionic curing accelerator that promotes the curing reaction by generating anions in response to irradiation with ultraviolet light or visible light or heating, or a cationic curing accelerator that promotes the curing reaction by generating cations in response to irradiation with ultraviolet light or visible light or heating is preferable.

Examples of the anionic curing accelerator include imidazoles such as 2-methylimidazole, 2-ethylimidazole, 2-ethyl-4-methylimidazole; trialkyl amines such as triethylamine, tributylamine; 4-dimethylaminopyridine, benzyldimethylamine, 2,4,6,-tris (dimethylaminomethyl) phenol, 1,8-diazabicyclo(5,4,0)-undecene, etc., and 4-dimethylaminopyridine and 1,8-diazabicyclo(5,4,0)-undecene are preferable. Additionally, phosphines such as triphenylphosphine; quaternary ammonium salts such as tetrabutylammonium salt, triisopropylmethylammonium salt, trimethyldecanylammonium salt, cetyltrimethylammonium salt, hexadecyltrimethylammonium hydroxide, etc. can be mentioned, but not limited to these. Furthermore, these may be used alone or in combination.

Examples of the cationic curing accelerator include quaternary phosphonium salts such as triphenylbenzylphosphonium salt, triphenylethylphosphonium salt, tetrabutylphosphonium salt (the counter ions of the quaternary salts are not specifically limited, but can include halogen, organic acid ions, hydroxide ions, etc., with organic acid ions and hydroxide ions being particularly preferable); transition metal compounds (transition metal salts) such as tin octanoate, zinc carboxylates (zinc 2-ethylhexanoate, zinc stearate, zinc behenate, zinc myristate), zinc phosphate esters (zinc octyl phosphate, zinc stearyl phosphate), etc., but not limited to these. Furthermore, these may be used alone or in combination.

The blending amount of the curing accelerator is used as required in the range of 0.01 parts by mass to 5.0 parts by mass per 100 parts by mass of the curable resin composition.

### [Inorganic Filler]

The curable resin composition of the present invention may include an inorganic filler. Examples of the inorganic filler include powders such as fused silica, crystalline silica, porous silica, alumina, zircon, calcium silicate, calcium carbonate, quartz powder, silicon carbide, silicon nitride, boron nitride, zirconia, aluminum nitride, graphite, forsterite, steatite, spinel, mullite, titania, talc, clay, iron oxide, asbestos, glass powder, or inorganic filler materials made by shaping these into spherical or crushed forms, but not limited to these. Furthermore, these may be used alone or in combination.

In the case of obtaining a curable resin composition for semiconductor encapsulation, the usage amount of the inorganic filler is preferably 80 parts by mass to 92 parts by mass, and more preferably 83 parts by mass to 90 parts by mass per 100 parts by mass of the curable resin composition. In addition, in the case of obtaining a curable resin composition for interlayer insulation layer formation materials and substrate materials such as copper-clad laminates, prepregs, and RCC, the usage amount of the above inorganic filler is preferably 5 parts by mass to 80 parts by mass, and more preferably 10 parts by mass to 60 parts by mass, per 100 parts by mass of the curable resin composition.

### [Polymerization Initiator]

The curable resin composition of the present invention can also improve curability by adding a polymerization initiator. The polymerization initiator refers to a compound capable of polymerizing olefin functional groups such as ethylenic unsaturated bonds, examples of which include olefin metathesis polymerization initiators, anionic polymerization initiators, cationic polymerization initiators, radical polymerization initiators, etc. Among these, it is preferable to use a radical polymerization initiator that has curability and moderate stability. The radical polymerization initiator refers to a compound that generates radicals by irradiation with ultraviolet light or visible light or by heating, and initiates a chain polymerization reaction. Examples of the radical polymerization initiator that can be used include organic peroxides, azo compounds, benzopinacol compounds, etc., and it is preferable to use organic peroxides due to control of curing temperature, suppression of outgassing, and less impact of decomposition products on electrical properties.

Examples of the above organic peroxides include ketone peroxides such as methyl ethyl ketone peroxide and acetylacetone peroxide; diacyl peroxides such as benzoyl peroxide; dialkyl peroxides such as dicumyl peroxide and 1,3-bis-(t-butylperoxyisopropyl)-benzene; peroxy ketals such as t-butyl peroxybenzoate and 1,1-di-t-butylperoxycyclohexane; alkyl peresters such as α-cumyl peroxyneodecanoate, t-butyl peroxyneodecanoate, t-butyl peroxypivalate, 1,1,3,3-tetramethylbutylperoxy-2-ethylhexanoate, t-amylperoxy-2-ethylhexanoate, t-butylperoxy-2-ethylhexanoate, t-amylperoxy-3,5,5-trimethylhexanoate, t-butylperoxy-3,5,5-trimethylhexanoate, and t-amylperoxybenzoate; peroxy carbonates such as di-2-ethylhexyl peroxydicarbonate, bis(4-t-butylcyclohexyl) peroxydicarbonate, t-butyl peroxyisopropyl carbonate, and 1,6-bis(t-butylperoxycarbonyloxy) hexane; t-butyl hydroperoxide, cumene hydroperoxide, t-butyl peroxyoctanoate, lauroyl peroxide, etc., but not limited to these. In addition, these may be used alone or in combination. Among the above organic peroxides, ketone peroxides, diacyl peroxides, hydroperoxides, dialkyl peroxides, peroxy ketals, alkyl peresters, peroxy carbonates, etc. are preferable, and dialkyl peroxides are more preferable.

Examples of the above azo compounds include azobisisobutyronitrile, 4,4′-azobis(4-cyanovaleric acid), 2,2′-azobis(2,4-dimethylvaleronitrile), etc., but not limited to these. These may be used alone or in combination.

The blending amount of the polymerization initiator is preferably 0.01 parts by mass to 5 parts by mass, and particularly preferably 0.01 parts by mass to 3 parts by mass, per 100 parts by mass of the curable resin composition. When the amount of the polymerization initiator used is less than 0.01 parts by mass, there is a risk that the molecular weight may not sufficiently increase during the polymerization reaction; and when the amount exceeds 5 parts by mass, there is a risk of impairing the dielectric properties such as dielectric constant and dielectric loss tangent. [Polymerization Inhibitor]

The curable resin composition of the present invention may include a polymerization inhibitor. By including the polymerization inhibitor, storage stability can be improved, and the reaction initiation temperature can be controlled. By controlling the reaction initiation temperature, it becomes easy to ensure flowability, and B-staging, such as prepregging, becomes easy without impairing impregnation into glass cloth or the like. When the polymerization reaction progresses excessively during prepregging, defects such as difficulty in lamination during the lamination process are likely to occur.

The polymerization inhibitor may be added either during the synthesis of the compound of the present invention or after the synthesis. The usage amount of the polymerization inhibitor is 0.008 parts by mass to 1 part by mass, preferably 0.01 parts by mass to 0.5 parts by mass, per 100 parts by mass of the compound of the present invention.

Examples of the polymerization inhibitor include phenolic, sulfur-based, phosphorus-based, hindered amine-based, nitroso-based, and nitroxyl radical-based polymerization inhibitors. In addition, the polymerization inhibitor may be used alone or in combination. Among these, in the present invention, phenolic, hindered amine-based, nitroso-based, and nitroxyl radical-based polymerization inhibitors are preferable.

Examples of the above phenolic polymerization inhibitor include monophenols such as 2,6-di-t-butyl-p-cresol, butylated hydroxyanisole, 2,6-di-t-butyl-p-ethylphenol, stearyl-β-(3,5-di-t-butyl-4-hydroxyphenyl) propionate, isooctyl-3-(3,5-di-t-butyl-4-hydroxyphenyl) propionate, 2,4-bis-(n-octylthio)-6-(4-hydroxy-3,5-di-t-butylanilino)-1,3,5-triazine, 2,4-bis[(octylthio)methyl]-o-cresol, etc.; bisphenols such as 2,2′-methylenebis(4-methyl-6-t-butylphenol), 2,2′-methylenebis(4-ethyl-6-t-butylphenol), 4,4′-thiobis(3-methyl-6-t-butylphenol), 4,4′-butylidenebis(3-methyl-6-t-butylphenol), triethylene glycol-bis[3-(3-t-butyl-5-methyl-4-hydroxyphenyl) propionate], 1,6-hexanediol-bis[3-(3,5-di-t-butyl-4-hydroxyphenyl) propionate], N,N′-hexamethylenebis(3,5-di-t-butyl-4-hydroxy-hydrocinnamide), 2,2-thio-diethylenebis[3-(3,5-di-t-butyl-4-hydroxyphenyl) propionate], 3,5-di-t-butyl-4-hydroxybenzylphosphonate-diethyl ester, 3,9-bis[1,1-dimethyl-2-{β-(3-t-butyl-4-hydroxy-5-methylphenyl) propionyloxy}ethyl]2,4,8,10-tetraoxaspiro[5,5]undecane, bis(3,5-di-t-butyl-4-hydroxybenzylsulfonic acid ethyl) calcium, etc.; high molecular weight phenols such as 1,1,3-tris(2-methyl-4-hydroxy-5-t-butylphenyl) butane, 1,3,5-trimethyl-2,4,6-tris(3,5-di-t-butyl-4-hydroxybenzyl)benzene, tetrakis-[methylene-3-(3′,5′-di-t-butyl-4′-hydroxyphenyl) propionate]methane, bis[3,3′-bis-(4′-hydroxy-3′-t-butylphenyl) butyric acid]glycol ester, tris-(3,5-di-t-butyl-4-hydroxybenzyl)-isocyanurate, 1,3,5-tris(3′,5′-di-t-butyl-4′-hydroxybenzyl)-S-triazine-2,4,6-(1H,3H,5H)trione, tocopherol, etc., but not limited to these.

Examples of the above sulfur-based polymerization inhibitor include dilauryl-3,3′-thiodipropionate, dimyristyl-3,3′-thiodipropionate, distearyl-3,3′-thiodipropionate, etc., but not limited to these.

Examples of the above phosphorus-based polymerization inhibitor include phosphites such as triphenyl phosphite, diphenyl isodecyl phosphite, phenyl diisodecyl phosphite, tris(nonylphenyl) phosphite, diisodecyl pentaerythritol phosphite, tris(2,4-di-t-butylphenyl) phosphite, cyclic neopentane tetraylbis(octadecyl)phosphite, cyclic neopentane tetraylbis(2,4-di-t-butylphenyl) phosphite, cyclic neopentane tetraylbis(2,4-di-t-butyl-4-methylphenyl) phosphite, bis[2-t-butyl-6-methyl-4-{2-(octadecyloxycarbonyl)ethyl}phenyl] hydrogen phosphite, etc.; oxaphosphanaphthalene oxides such as 9,10-dihydro-9-oxa-10-phosphaphenanthrene-10-oxide, 10-(3,5-di-t-butyl-4-hydroxybenzyl)-9,10-dihydro-9-oxa-10-phosphaphenanthrene-10-oxide, 10-decyloxy-9,10-dihydro-9-oxa-10-phosphaphenanthrene-10-oxide, etc., but not limited to these.

Examples of the above hindered amine-based polymerization inhibitor include ADEKASTAB LA-40MP, ADEKASTAB LA-40Si, ADEKASTAB LA-402AF, ADEKASTAB LA-87, ADEKASTAB LA-82, ADEKASTAB LA-81, ADEKASTAB LA-77Y, ADEKASTAB LA-77G, ADEKASTAB LA-72, ADEKASTAB LA-68, ADEKASTAB LA-63P, ADEKASTAB LA-57, ADEKASTAB LA-52, Chimassorb 2020FDL, Chimassorb 944FDL, Chimassorb 944LD, Tinuvin 622SF, Tinuvin PA144, Tinuvin 765, Tinuvin 770DF, Tinuvin XT55FB, Tinuvin 111FDL, Tinuvin 783FDL, Tinuvin 791FB, etc., but not limited to these.

Examples of the above nitroso-based polymerization inhibitor include p-nitrosophenol, N-nitrosodiphenylamine, ammonium salt of N-nitrosophenylhydroxylamine (cupferron), etc., but not limited to these. Among these, the ammonium salt of N-nitrosophenylhydroxylamine (cupferron) is preferable.

Examples of the above nitroxyl radical-based polymerization inhibitor include di-tert-butyl nitroxide, 2,2,6,6-tetramethylpiperidine-1-oxyl, 4-hydroxy-2,2,6,6-tetramethylpiperidine-1-oxyl, 4-oxo-2,2,6,6-tetramethylpiperidine-1-oxyl, 4-amino-2,2,6,6-tetramethylpiperidine-1-oxyl, 4-methoxy-2,2,6,6-tetramethylpiperidine-1-oxyl, 4-acetoxy-2,2,6,6-tetramethylpiperidine-1-oxyl, 4-benzoyloxy-2,2,6,6-tetramethylpiperidine-1-oxyl, etc., but not limited to these.

### [Flame Retardant]

The curable resin composition of the present invention may use a flame retardant. Examples of the flame retardant include halogen-based flame retardants, inorganic flame retardants (antimony compounds, metal hydroxides, nitrogen compounds, boron compounds, etc.), phosphorus-based flame retardants, etc., but phosphorus-based flame retardants are preferable from the viewpoint of achieving halogen-free flame retardancy.

The above phosphorus-based flame retardants may be either reactive or additive types. Specific examples include phosphate esters such as trimethyl phosphate, triethyl phosphate, tricresyl phosphate, trixylylenyl phosphate, cresyl diphenyl phosphate, cresyl-2,6-dixylylenyl phosphate, 1,3-phenylene bis(dixylylenyl phosphate), 1,4-phenylene bis(dixylylenyl phosphate), 4,4′-biphenyl(dixylylenyl phosphate), etc.; phosphanes such as 9,10-dihydro-9-oxa-10-phosphaphenanthrene-10-oxide, 10 (2,5-dihydroxyphenyl)-10H-9-oxa-10-phosphaphenanthrene-10-oxide, etc.; phosphorus-containing epoxy compounds obtained by reacting epoxy resins with active hydrogen of the aforementioned phosphanes; red phosphorus, etc., but not limited to these. In addition, these may be used alone or in combination. Among the above exemplary substances, phosphate esters, phosphanes, or phosphorus-containing epoxy compounds are preferable, and 1,3-phenylene bis(dixylylenyl phosphate), 1,4-phenylene bis(dixylylenyl phosphate), 4,4′-biphenyl(dixylylenyl phosphate), or phosphorus-containing epoxy compounds are particularly preferable.

The content of the flame retardant is preferably in the range of 0.1 parts by mass to 10 parts by mass per 100 parts by mass of the curable resin composition. When the content is less than 0.1 parts by mass, there is a risk that the flame retardancy may be insufficient; and when the content is more than 10 parts by mass, there is a risk of adversely affecting the moisture absorption and dielectric properties of the cured product.

### [Light Stabilizer]

The curable resin composition of the present invention may use a light stabilizer. Hindered amine-based light stabilizers, especially HALS, are suitable as the light stabilizer. Examples of the HALS include a reaction product of dibutylamine, 1,3,5-triazine, N,N′-bis(2,2,6,6-tetramethyl-4-piperidyl-1,6-hexamethylenediamine and N-(2,2,6,6-tetramethyl-4-piperidyl)butylamine; a reaction product of dimethyl succinate and 1-(2-hydroxyethyl)-4-hydroxy-2,2,6,6-tetramethylpiperidine; poly[{6-(1,1,3,3-tetramethylbutyl)amino-1,3,5-triazine-2,4-diyl}{(2,2,6,6-tetramethyl-4-piperidyl)imino}hexamethylene {(2,2,6,6-tetramethyl-4-piperidyl)imino}]; bis(1,2,2,6,6-pentamethyl-4-piperidyl) [[3,5-bis(1,1-dimethylethyl)-4-hydroxyphenyl]methyl] butylmalonate; bis(2,2,6,6-tetramethyl-4-piperidyl) sebacate; bis(1,2,2,6,6-pentamethyl-4-piperidyl) sebacate; bis(1-octyloxy-2,2,6,6-tetramethyl-4-piperidyl) sebacate; bis(1,2,2,6,6-pentamethyl-4-piperidyl) 2-(3,5-di-t-butyl-4-hydroxybenzyl)-2-n-butylmalonate, etc., but not limited to these. In addition, these may be used alone or in combination.

The content of the light stabilizer is preferably in the range of 0.001 parts by mass to 0.1 parts by mass per 100 parts by mass of the curable resin composition. When the content is less than 0.001 parts by mass, there is a risk that it may be insufficient to exhibit the light stabilizing effect; and when the content is more than 0.1 parts by mass, there is a risk of adversely affecting the moisture absorption and dielectric properties of the cured product.

### [Binder Resin]

The curable resin composition of the present invention may use a binder resin. Examples of the binder resin include butyral resins, acetal resins, acrylic resins, epoxy-nylon resins, NBR-phenolic resins, epoxy-NBR resins, silicone resins, etc., but not limited to these. In addition, these may be used alone or in combination.

The blending amount of the binder resin is preferably in a range that does not impair the flame retardancy and heat resistance of the cured product, and is preferably 0.05 parts by mass to 50 parts by mass per 100 parts by mass of the curable resin composition, and more preferably 0.05 parts by mass to 20 parts by mass is used as required.

### [Additives]

The curable resin composition of the present invention may include additives. Examples of the additives include modified products of acrylonitrile copolymers, polyethylene, fluorine resins, silicone gels, silicone oils, surface treatment agents for fillers such as silane coupling agents, release agents, colorants such as carbon black, phthalocyanine blue, and phthalocyanine green.

The blending amount of the additives is preferably 1 part by mass or less, more preferably 0.7 parts by mass or less, per 100 parts by mass of the curable resin composition.

The curable resin composition of the present invention may further use epoxy resins, active ester compounds, phenolic resins, polyphenylene ether compounds, amine resins, compounds having ethylenic unsaturated bonds, isocyanate resins, polyamide resins, cyanate ester resins, polyimide resins, polybutadiene and modified products thereof, polystyrene and modified products thereof, polyethylene and modified products thereof, etc., and these may be used alone or in combination. Among these compounds, it is preferable to include polyphenylene ether compounds, compounds having ethylenic unsaturated bonds, maleimide compounds, cyanate ester resins, polybutadiene and modified products thereof, and polystyrene and modified products thereof, in consideration of the balance of heat resistance, adhesion, and dielectric properties. By including these compounds, the brittleness of the cured product can be improved and the adhesion to metals can be enhanced, thereby suppressing package cracking in reliability tests such as solder reflow and thermal cycling. Unless otherwise specified, the total usage amount of these compounds is preferably 10 parts by mass or less, more preferably 5 parts by mass or less, and even more preferably 3 parts by mass or less, with respect to the compound of the present invention. Further, the preferable lower limit value is 0.1 parts by mass more, more preferably 0.25 parts by mass or more, and even more preferably 0.5 parts by mass or more. By setting the usage amount within the above range, the effects of heat resistance and dielectric properties of the compound of the present invention can be utilized while adding the effects of each added compound. The following examples can be used for these components.

### [Epoxy Resin]

The following are preferable examples of the epoxy resins, but not limited to these. The properties of the epoxy resins may be liquid or solid, and the epoxy resins may be used alone or in combination.

Examples of the liquid epoxy resins include bisphenol A type epoxy resins, bisphenol F type epoxy resins, bisphenol AF type epoxy resins, naphthalene type epoxy resins, glycidyl ester type epoxy resins, glycidyl amine type epoxy resins, phenol novolac type epoxy resins, alicyclic epoxy resins having an ester skeleton, cyclohexane type epoxy resins, cyclohexane dimethanol type epoxy resins, glycidyl amine type epoxy resins, and epoxy resins having a butadiene structure. Specific examples include “RE310S”, “RE410S” (both manufactured by Nippon Kayaku, bisphenol A type epoxy resins), “RE303S”, “RE304S”, “RE403S”, “RE404S” (all manufactured by Nippon Kayaku, bisphenol F type epoxy resins), “HP4032”, “HP4032D”, “HP4032SS” (all manufactured by DIC, naphthalene type epoxy resins), “828US”, “jER828EL”, “825”, “828EL” (all manufactured by Mitsubishi Chemical, bisphenol A type epoxy resins), “jE807”, “1750” (both manufactured by Mitsubishi Chemical, bisphenol F type epoxy resins), “jER152” (manufactured by Mitsubishi Chemical, phenol novolac type epoxy resin), “630”, “630LSD” (both manufactured by Mitsubishi Chemical, glycidyl amine type epoxy resins), “ZX1059” (manufactured by Nippon Steel & Sumikin Chemical, mixture of bisphenol A type epoxy resin and bisphenol F type epoxy resin), “EX-721” (manufactured by Nagase ChemteX, glycidyl ester type epoxy resin), “Celloxide 2021P” (manufactured by Daicel, alicyclic epoxy resin having an ester skeleton), “PB-3600” (manufactured by Daicel, epoxy resin having a butadiene structure), “ZX1658”, “ZX1658GS” (both manufactured by Nippon Steel & Sumikin Chemical, liquid 1,4-glycidyl cyclohexane type epoxy resins), etc. These may be used alone or in combination of two or more.

Preferable examples of the solid epoxy resins include bixylylene type epoxy resins, naphthalene type epoxy resins, naphthalene type tetrafunctional epoxy resins, cresol novolac type epoxy resins, dicyclopentadiene type epoxy resins, trisphenol type epoxy resins, naphthol type epoxy resins, biphenyl type epoxy resins, naphthalene ether type epoxy resins, anthracene type epoxy resins, bisphenol A type epoxy resins, bisphenol AF type epoxy resins, and tetraphenylethane type epoxy resins. Naphthol type epoxy resins, bisphenol AF type epoxy resins, naphthalene type epoxy resins, and biphenyl type epoxy resins can be mentioned.

Specific examples include “HP4032H” (manufactured by DIC, naphthalene type epoxy resin), “HP-4700”, “HP-4710” (both manufactured by DIC, naphthalene type tetrafunctional epoxy resins), “N-690” (manufactured by DIC, cresol novolac type epoxy resin), “N-695” (manufactured by DIC, cresol novolac type epoxy resin), “HP-7200” (manufactured by DIC, dicyclopentadiene type epoxy resin), “HP-7200”, “HP-7200HH”, “HP-7200H” (all manufactured by DIC, dicyclopentadiene type epoxy resins), “EXA-7311”, “EXA-7311-G3”, “EXA-7311-G4”, “EXA-7311-G4S”, “HP-6000” (all manufactured by DIC, naphthalene ether type epoxy resins), “EPPN-502H” (manufactured by Nippon Kayaku, trisphenol type epoxy resin), “NC-7000L”, “NC-7300” (both manufactured by Nippon Kayaku, naphthol-cresol novolac type epoxy resins), “NC-3000H”, “NC-3000”, “NC-3000L”, “NC-3100” (all manufactured by Nippon Kayaku, biphenyl aralkyl type epoxy resins), “XD-1000-2L”, “XD-1000-L”, “XD-1000-H”, “XD-1000-H” (all manufactured by Nippon Kayaku, dicyclopentadiene type epoxy resins), “ESN475V” (manufactured by Nippon Steel & Sumikin Chemical, naphthol type epoxy resin), “ESN485” (manufactured by Nippon Steel & Sumikin Chemical, naphthol novolac type epoxy resin), “YX-4000H”, “YX-4000”, “YL6121” (all manufactured by Mitsubishi Chemical, biphenyl type epoxy resins), “YX-4000HK” (manufactured by Mitsubishi Chemical, bixylylene type epoxy resin), “YX-8800” (manufactured by Mitsubishi Chemical, anthracene type epoxy resin), “PG-100”, “CG-500” (manufactured by Osaka Gas Chemicals, fluorene-based epoxy resins), “YL-7760” (manufactured by Mitsubishi Chemical, bisphenol AF type epoxy resin), “YL-7800” (manufactured by Mitsubishi Chemical, fluorene type epoxy resin), “jER1010” (manufactured by Mitsubishi Chemical, solid bisphenol A type epoxy resin), “jER1031S” (manufactured by Mitsubishi Chemical, tetraphenylethane type epoxy resin), etc. These may be used alone or in combination of two or more.

### [Active Ester Compound]

The active ester compound refers to a compound that includes at least one ester bond in the structure, and has aliphatic chains, aliphatic rings, or aromatic rings bonded on both sides of the ester bond. Examples of the active ester compounds include compounds having two or more highly reactive ester groups in one molecule, such as phenol esters, thiophenol esters, N-hydroxyamine esters, and esters of heterocyclic hydroxy compounds. These can be obtained by a condensation reaction of at least one compound of carboxylic acid compounds, acid chlorides, or thiocarboxylic acid compounds with at least one compound of hydroxy compounds or thiol compounds. Particularly, from the viewpoint of improving heat resistance, it is preferable to obtain these from carboxylic acid compounds or acid chlorides and hydroxy compounds, and as the hydroxy compounds, phenol compounds or naphthol compounds are preferable. The active ester compounds may be used alone or in combination of two or more.

Examples of the above carboxylic acid compounds include benzoic acid, acetic acid, succinic acid, maleic acid, itaconic acid, phthalic acid, isophthalic acid, terephthalic acid, pyromellitic acid, etc.

Examples of the above acid chlorides include acetyl chloride, acryloyl chloride, methacryloyl chloride, malonyl chloride, succinyl dichloride, diglycolyl chloride, glutaryl dichloride, suberoyl dichloride, sebacoyl dichloride, adipoyl dichloride, dodecanedioyl dichloride, azelaoyl chloride, 2,5-furandicarbonyl dichloride, phthaloyl chloride, isophthaloyl chloride, terephthaloyl chloride, trimesic acid chloride, bis(4-chlorocarbonylphenyl) ether, 4,4′-diphenyldicarbonyl chloride, 4,4′-azodibenzoyl dichloride, etc.

Examples of the above phenol compounds and the above naphthol compounds include hydroquinone, resorcinol, bisphenol A, bisphenol F, bisphenol S, phenolphthalein, methylated bisphenol A, methylated bisphenol F, methylated bisphenol S, phenol, o-cresol, m-cresol, p-cresol, catechol, α-naphthol, β-naphthol, 1,5-dihydroxynaphthalene, 1,6-dihydroxynaphthalene, 2,6-dihydroxynaphthalene, dihydroxybenzophenone, trihydroxybenzophenone, tetrahydroxybenzophenone, phloroglucinol, benzenetriol, dicyclopentadiene type diphenol compounds, phenol novolac, phenolic resins to be described later, etc. Here, “dicyclopentadiene type diphenol compound” refers to a diphenol compound obtained by condensing two phenol molecules with one dicyclopentadiene molecule.

Preferable specific examples of the active ester compounds include active ester compounds containing a dicyclopentadiene type diphenol structure, active ester compounds containing a naphthalene structure, active ester compounds containing an acetylated product of phenol novolac, active ester compounds containing a benzoylated product of phenol novolac, the compound described in Example 2 of International Publication No. 2020/095829, compounds disclosed in International Publication No. 2020/059625, etc. Among these, active ester compounds containing a naphthalene structure and active ester compounds containing a dicyclopentadiene type diphenol structure are more preferable. The dicyclopentadiene type diphenol structure refers to a divalent structural unit composed of phenylene-dicyclopentylene-phenylene.

As commercially available active ester compounds, for example, “EXB9451”, “EXB9460”, “EXB9460S”, “HPC-8000-65T”, “HPC-8000H-65TM”, “EXB-8000L-65TM”, “EXB-8150-65T” (manufactured by DIC) can be mentioned as active ester compounds containing a dicyclopentadiene type diphenol structure; “EXB9416-70BK” (manufactured by DIC) can be mentioned as an active ester compound containing a naphthalene structure; “DC808” (manufactured by Mitsubishi Chemical) can be mentioned as an active ester compound containing an acetylated product of phenol novolac; “YLH1026”, “YLH1030”, “YLH1048” (manufactured by Mitsubishi Chemical) can be mentioned as active ester compounds containing a benzoylated product of phenol novolac; “DC808” (manufactured by Mitsubishi Chemical) can be mentioned as an active ester-based curing agent which is an acetylated product of phenol novolac; and “EXB-9050L-62M” manufactured by DIC can be mentioned as a phosphorus atom-containing active ester-based curing agent.

### [Phenolic Resin]

The phenolic resin refers to a compound having two or more phenolic hydroxyl groups in the molecule. Examples of the phenolic resins include reaction products of phenols and aldehydes, reaction products of phenols and diene compounds, reaction products of phenols and ketones, reaction products of phenols and substituted biphenyls, reaction products of phenols and substituted phenyls, reaction products of bisphenols and aldehydes, etc., but not limited to these.

In addition, these may be used alone or in combination.

Specific examples of the above raw materials are exemplified below, but not limited to these.

### <Phenols>

Phenol, alkyl-substituted phenols, aromatic-substituted phenols, hydroquinone, resorcinol, naphthol, alkyl-substituted naphthols, dihydroxybenzene, alkyl-substituted dihydroxybenzenes, dihydroxynaphthalenes, etc.

### <Aldehydes>

Formaldehyde, acetaldehyde, alkyl aldehydes, benzaldehyde, alkyl-substituted benzaldehydes, hydroxybenzaldehyde, naphthaldehyde, glutaraldehyde, phthalaldehyde, crotonaldehyde, cinnamaldehyde, furfural, etc.

### <Diene Compound>

Dicyclopentadiene, terpenes, vinylcyclohexene, norbornadiene, vinylnorbornene, tetrahydroindene, divinylbenzene, divinylbiphenyl, diisopropenylbiphenyl, butadiene, isoprene, etc.

### <Ketones>

Acetone, methyl ethyl ketone, methyl isobutyl ketone, acetophenone, benzophenone, fluorenone, etc.

### <Substituted Biphenyls>

4,4′-bis(chloromethyl)-1,1′-biphenyl, 4,4′-bis(methoxymethyl)-1,1′-biphenyl, 4,4′-bis(hydroxymethyl)-1,1′-biphenyl, etc.

### <Substituted Phenyls>

1,4-bis(chloromethyl)benzene, 1,4-bis(methoxymethyl)benzene, 1,4-bis(hydroxymethyl)benzene, etc.

### [Polyphenylene Ether Compound]

As the polyphenylene ether compound, from the viewpoint of heat resistance and electrical properties, it is preferable to use a polyphenylene ether compound having an ethylenic unsaturated bond, and it is more preferable to use a polyphenylene ether compound having an acrylic group, a methacrylic group, or a styrene structure. Commercially available products include SA-9000 (manufactured by SABIC, a polyphenylene ether compound having a methacrylic group), OPE-2St 1200 (manufactured by Mitsubishi Gas Chemical, a polyphenylene ether compound having a styrene structure), or the like.

The number average molecular weight (Mn) of the polyphenylene ether compound is preferably 500 to 5000, more preferably 2000 to 5000, and even more preferably 2000 to 4000. When the molecular weight is less than 500, there is a tendency that sufficient heat resistance of the cured product cannot be obtained. In addition, when the molecular weight is more than 5000, the melt viscosity becomes high, and sufficient flowability cannot be obtained, which tends to result in molding defects. Furthermore, the reactivity also decreases, requiring a longer time for the curing reaction, and the amount of unreacted material that is not incorporated into the curing system increases, resulting in a tendency for the glass transition temperature of the cured product to decrease and the heat resistance of the cured product to deteriorate.

If the number average molecular weight of the polyphenylene ether compound is 500 to 5000, it is possible to maintain excellent dielectric properties while exhibiting excellent heat resistance and moldability. It should be noted that the number average molecular weight mentioned here can be specifically measured using gel permeation chromatography or the like.

The polyphenylene ether compound may be obtained by a polymerization reaction, or may be obtained by a redistribution reaction of a high molecular weight polyphenylene ether compound with a number average molecular weight of about 10000 to 30000. Additionally, with these as raw materials, radical polymerizability may be imparted by reacting with compounds having ethylenic unsaturated bonds such as methacryloyl chloride, acryloyl chloride, or chloromethylstyrene. The polyphenylene ether compound obtained by a redistribution reaction can be obtained, for example, by heating a high molecular weight polyphenylene ether compound in a solvent such as toluene in the presence of a phenolic compound and a radical initiator to undergo redistribution reaction. The polyphenylene ether compound obtained by such redistribution reaction is preferable because the compound can maintain even higher heat resistance due to having hydroxyl groups derived from phenolic compounds that contribute to curing at both ends of the molecular chain, and functional groups can be introduced at both ends of the molecular chain even after modification with compounds having ethylenic unsaturated bonds. Moreover, the polyphenylene ether compound obtained by a polymerization reaction is preferable in terms of exhibiting excellent flowability.

The adjustment of the molecular weight of the polyphenylene ether compound can be performed by adjusting polymerization conditions or the like in the case of polyphenylene ether compounds obtained by a polymerization reaction. Moreover, in the case of polyphenylene ether compounds obtained by a redistribution reaction, the molecular weight of the polyphenylene ether compound obtained can be adjusted by adjusting the conditions of the redistribution reaction or the like. More specifically, it is considered to adjust the blending amount of the phenolic compound used in the redistribution reaction. In other words, the higher the blending amount of the phenolic compound, the lower the molecular weight of the polyphenylene ether compound obtained. In this case, poly(2,6-dimethyl-1,4-phenylene ether) or the like can be used as the high molecular weight polyphenylene ether compound that undergoes the redistribution reaction. In addition, although not particularly limited, the phenolic compound used in the aforementioned redistribution reaction preferably includes, for example, multifunctional phenolic compounds having two or more phenolic hydroxyl groups in the molecule, such as bisphenol A, phenol novolac, and cresol novolac. These may be used alone or in combination of two or more.

### [Amine Resin]

The amine resin refers to a compound having two or more amino groups in the molecule. Examples of the amine resins include diaminodiphenylmethane, diaminodiphenylsulfone, isophorone diamine, naphthalene diamine, aniline novolac (reaction product of aniline and formalin), N-methylaniline novolac (reaction product of N-methylaniline and formalin), ortho-ethylaniline novolac (reaction product of ortho-ethylaniline and formalin), reaction product of 2-methylaniline and formalin, reaction product of 2,6-diisopropylaniline and formalin, reaction product of 2,6-diethylaniline and formalin, reaction product of 2-ethyl-6-ethylaniline and formalin, reaction product of 2,6-dimethylaniline and formalin, aniline resin obtained by the reaction of aniline and xylylene chloride, reaction product of aniline and substituted biphenyl compounds (4,4′-bis(chloromethyl)-1,1′-biphenyl and 4,4′-bis(methoxymethyl)-1,1′-biphenyl, etc.) as described in Japanese Patent No. 6429862, reaction product of aniline and substituted phenyl 1,4-compounds (1,4-bis(chloromethyl)benzene, 1,4-bis(methoxymethyl)benzene and 1 bis(hydroxymethyl)benzene, etc.), 4,4′-(1,3-phenylenediisopropylidene)bisaniline, 4,4′-(1,4-phenylenediisopropylidene)bisaniline, reaction product of aniline and diisopropenylbenzene, dimer diamine, etc., but not limited to these. In addition, these may be used alone or in combination.

### [Compound Containing Ethylenic Unsaturated Bonds]

The compound containing ethylenic unsaturated bonds refers to a compound having one or more ethylenic unsaturated bonds in the molecule that can be polymerized by heat or light, regardless of whether a polymerization initiator is used or not.

Examples of the compounds containing ethylenic unsaturated bonds include reaction products of the aforementioned phenolic resins and halogen-based compounds containing ethylenic unsaturated bonds (such as chloromethylstyrene, allyl chloride, methallyl chloride, acryloyl chloride, methacryloyl chloride, etc.), reaction products of phenols containing ethylenic unsaturated bonds (such as 2-allylphenol, 2-propenylphenol, 4-allylphenol, 4-propenylphenol, eugenol, isoeugenol, etc.) and halogen-based compounds (such as 1,4-bis(chloromethyl)benzene, 4,4′-bis(chloromethyl) biphenyl, 4,4′-difluorobenzophenone, 4,4′-dichlorobenzophenone, 4,4′-dibromobenzophenone, cyanuric chloride, etc.), reaction products of epoxy resins or alcohols with (meth)acrylic acids (such as acrylic acid, methacrylic acid, etc.) and acid-modified products thereof, etc., but not limited to these. In addition, these may be used alone or in combination.

### [Isocyanate Resin]

The isocyanate resin refers to a compound having two or more isocyanate groups in the molecule. Examples of the isocyanate resins include aromatic diisocyanates such as p-phenylene diisocyanate, m-phenylene diisocyanate, p-xylylene diisocyanate, m-xylylene diisocyanate, 2,4-tolylene diisocyanate, 2,6-tolylene diisocyanate, 4,4′-diphenylmethane diisocyanate, naphthalene diisocyanate; aliphatic or alicyclic diisocyanates such as isophorone diisocyanate, hexamethylene diisocyanate, 4,4′-dicyclohexylmethane diisocyanate, hydrogenated xylylene diisocyanate, norbornene diisocyanate, lysine diisocyanate; polyisocyanates such as biuret compounds of one or more isocyanate monomers, or trimerized isocyanate compounds of the above diisocyanate compounds; polyisocyanates obtained by urethane reaction between the above isocyanate compounds and polyol compounds, etc., but not limited to these. In addition, these compounds may be used alone or in combination.

### [Polyamide Resin]

Examples of the polyamide resins include reaction products of one or more of diamine, diisocyanate, oxazoline with dicarboxylic acid, reaction products of diamine and acid chloride, and ring-opening polymerization products of lactam compounds. In addition, these may be used alone or in combination.

Specific examples of the above raw materials are exemplified below, but not limited to these.

### <Diamine>

Ethylenediamine, trimethylenediamine, tetramethylenediamine, pentamethylenediamine, hexamethylenediamine, heptamethylenediamine, octamethylenediamine, nonamethylenediamine, decanediamine, undecanediamine, dodecanediamine, tridecanediamine, tetradecanediamine, pentadecanediamine, hexadecanediamine, heptadecanediamine, octadecanediamine, nonadecanediamine, eicosanediamine, 2-methyl-1,5-diaminopentane, 2-methyl-1,8-diaminooctane, dimer diamine, cyclohexanediamine, bis(4-aminocyclohexyl) methane, bis(3-methyl-4-aminocyclohexyl) methane, xylylenediamine, norbornanediamine, isophoronediamine, bisaminomethyltricyclodecane, phenylenediamine, diethyltoluenediamine, naphthalenediamine, diaminodiphenylmethane, bis(4-amino-3,5-dimethylphenyl) methane, bis(4-amino-3,5-diethylphenyl) methane, 4,4′-methylenebis-o-toluidine, 4,4′-methylenebis-o-ethylaniline, 4,4′-methylenebis-2-ethyl-6-methylaniline, 4,4′-methylenebis-2,6-diisopropylaniline, 4,4-ethylenedianiline, diaminodiphenylsulfone, diaminodiphenyl ether, 1,3-bis(3-aminophenoxy)benzene, 1,3-bis(4-aminophenoxy)benzene, 4,4-bis(4-aminophenoxy) biphenyl, 2,2-bis[4-(4-aminophenoxy)phenyl] propane, bis[4-(4-aminophenoxy)phenyl] sulfone, 2,2-bis(3-amino-4-hydroxyphenyl) propane, 2,2-bis(4-aminophenyl) hexafluoropropane, 4,4′-(1,3-phenylenediisopropylidene)bisaniline, 4,4′-(1,4-phenylenediisopropylidene)bisaniline, 9,9-bis(4-aminophenyl) fluorene, 2,7-diaminofluorene, aminobenzylamine, diaminobenzophenone, etc.

### <Diisocyanate>

Benzene diisocyanate, toluene diisocyanate, 1,3-bis(isocyanatomethyl)benzene, 1,3-bis(isocyanatomethyl)cyclohexane, bis(4-isocyanatophenyl) methane, isophorone diisocyanate, 1,3-bis(2-isocyanato-2-propyl)benzene, 2,2-bis(4-isocyanatophenyl) hexafluoropropane, dicyclohexylmethane-4,4′-diisocyanate, etc.

### <Dicarboxylic Acid>

Oxalic acid, malonic acid, succinic acid, glutaric acid, adipic acid, suberic acid, azelaic acid, sebacic acid, undecanedioic acid, dodecanedioic acid, terephthalic acid, isophthalic acid, 5-hydroxyisophthalic acid, 2-chloroterephthalic acid, 2-methylterephthalic acid, 5-methylisophthalic acid, 5-sodium sulfoisophthalic acid, hexahydroterephthalic acid, hexahydroisophthalic acid, cyclohexanedicarboxylic acid, biphenyldicarboxylic acid, naphthalenedicarboxylic acid, benzophenonedicarboxylic acid, furandicarboxylic acid, 4,4′-dicarboxydiphenyl ether, 4,4′-dicarboxydiphenyl sulfide, etc.

### <Acid Chloride>

Acetyl chloride, acryloyl chloride, methacryloyl chloride, malonyl chloride, succinyl dichloride, diglycolyl chloride, glutaryl dichloride, suberoyl dichloride, sebacoyl dichloride, adipoyl dichloride, dodecanedioyl dichloride, azelaoyl chloride, 2,5-furandicarbonyl dichloride, phthaloyl chloride, isophthaloyl chloride, terephthaloyl chloride, trimesic acid chloride, bis(4-chlorocarbonylphenyl) ether, 4,4′-diphenyldicarbonyl chloride, 4,4′-azodibenzoyl dichloride, etc.

### <Lactam>

ε-caprolactam, ω-undecanelactam, ω-laurolactam, etc.

### [Polyimide Resin]

Examples of the polyimide resins include reaction products of the aforementioned diamine and tetracarboxylic dianhydrides exemplified below, but not limited to these. In addition, these may be used alone or in combination.

### <Tetracarboxylic Dianhydride>

4,4′-(hexafluoroisopropylidene)diphthalic anhydride, 5-(2,5-dioxotetrahydro-3-furanyl)-3-methyl-cyclohexene-1,2dicarboxylic anhydride, pyromellitic dianhydride, 1,2,3,4-benzenetetracarboxylic dianhydride, 3,3′,4,4′-benzophenonetetracarboxylic dianhydride, 2,2′,3,3′-benzophenonetetracarboxylic dianhydride, 3,3′,4,4′-biphenyltetracarboxylic dianhydride, 3,3′,4,4′-diphenylsulfonetetracarboxylic dianhydride, 2,2′,3,3′-biphenyltetracarboxylic dianhydride, methylene-4,4′-diphthalic dianhydride, 1,1-ethylidene-4,4′-diphthalic dianhydride, 2,2′-propylidene-4,4′-diphthalic dianhydride, 1,2-ethylene-4,4′-diphthalic dianhydride, 1,3-trimethylene-4,4′-diphthalic dianhydride, 1,4-tetramethylene-4,4′-diphthalic dianhydride, 1,5-pentamethylene-4,4′-diphthalic dianhydride, 4,4′-oxydiphthalic dianhydride, thio-4,4′-diphthalic dianhydride, sulfonyl-4,4′-diphthalic dianhydride, 1,3-bis(3,4-dicarboxyphenyl)benzene dianhydride, 1,3-bis(3,4-dicarboxyphenoxy)benzene dianhydride, 1,4-bis(3,4-dicarboxyphenoxy)benzene dianhydride, 1,3-bis[2-(3,4-dicarboxyphenyl)-2-propyl]benzene dianhydride, 1,4-bis[2-(3,4-dicarboxyphenyl)-2-propyl]benzene dianhydride, bis[3-(3,4-dicarboxyphenoxy)phenyl] methane dianhydride, bis[4-(3,4-dicarboxyphenoxy)phenyl] methane dianhydride, 2,2-bis[3-(3,4-dicarboxyphenoxy)phenyl] propane dianhydride, 2,2-bis[4-(3,4-dicarboxyphenoxy)phenyl] propane dianhydride, bis(3,4-dicarboxyphenoxy)dimethylsilane dianhydride, 1,3-bis(3,4-dicarboxyphenyl)-1,1,3,3-tetramethyldisiloxane dianhydride, 2,3,6,7-naphthalenetetracarboxylic dianhydride, 1,4,5,8-naphthalenetetracarboxylic dianhydride, 1,2,5,6-naphthalenetetracarboxylic dianhydride, 3,4,9,10-perylenetetracarboxylic dianhydride, 2,3,6,7-anthracenetetracarboxylic dianhydride, 1,2,7,8-phenanthrenetetracarboxylic dianhydride, ethylenetetracarboxylic dianhydride, 1,2,3,4-butanetetracarboxylic dianhydride, 1,2,3,4-cyclobutanetetracarboxylic dianhydride), cyclopentanetetracarboxylic dianhydride, cyclohexane-1,2,3,4-tetracarboxylic dianhydride, cyclohexane-1,2,4,5-tetracarboxylic dianhydride, 3,3′,4,4′-bicyclohexyltetracarboxylic dianhydride, carbonyl-4,4′-bis(cyclohexane-1,2-dicarboxylic acid)dianhydride, methylene-4,4′-bis(cyclohexane-1,2-dicarboxylic acid)dianhydride, 1,2-ethylene-4,4′-bis(cyclohexane-1,2-dicarboxylic acid)dianhydride, 1,1-ethylidene-4,4′-bis(cyclohexane-1,2-dicarboxylic acid)dianhydride, 2,2-propylidene-4,4′-bis(cyclohexane-1,2-dicarboxylic acid)dianhydride, oxy-4,4′-bis(cyclohexane-1,2-dicarboxylic acid)dianhydride, thio-4,4′-bis(cyclohexane-1,2-dicarboxylic acid)dianhydride, sulfonyl-4,4′-bis(cyclohexane-1,2-dicarboxylic acid)dianhydride, bicyclo[2,2,2]oct-7-ene-2,3,5,6-tetracarboxylic dianhydride, rel-[1S,5R,6R]-3-oxabicyclo[3,2,1]octane-2,4-dione-6-spiro-3′-(tetrahydrofuran-2′,5′-dione), 4-(2,5-dioxotetrahydrofuran-3-yl)-1,2,3,4-tetrahydronaphthalene-1,2-dicarboxylic anhydride, ethylene glycol-bis-(3,4-dicarboxylic anhydride phenyl) ether, 4,4′-biphenylbis(trimellitic acid monoester acid anhydride), 9,9′-bis(3,4-dicarboxyphenyl) fluorene dianhydride, etc.

### [Maleimide Compound]

The curable resin composition of the present invention may include a maleimide compound. The maleimide compound refers to a compound having one or more maleimide groups in the molecule. Examples of the maleimide compounds include 4,4′-diphenylmethane bismaleimide, polyphenylmethane maleimide, m-phenylene bismaleimide, 2,2′-bis[4-(4-maleimidophenoxy)phenyl] propane, 3,3′-dimethyl-5,5′-diethyl-4,4′-diphenylmethane bismaleimide, 4-methyl-1,3-phenylene bismaleimide, 4,4′-diphenyl ether bismaleimide, 4,4′-diphenylsulfone bismaleimide, 1,3-bis(3-maleimidophenoxy)benzene, 1,3-bis(4-maleimidophenoxy)benzene), xylok-type maleimide compound (Anilix Maleimide, manufactured by Mitsui Chemicals Fine), biphenyl aralkyl-type maleimide compound (solidified by removing the solvent under reduced pressure from the resin solution containing the maleimide compound (M2) described in Example 4 of Japanese Patent Application Laid-Open No. 2009-001783), bisaminocumylbenzene-type maleimide (maleimide compound described in International Publication No. 2020/054601), maleimide compounds having an indane structure described in Japanese Patent No. 6629692 or International Publication No. 2020/217679, maleimide compounds described in MATERIAL STAGE Vol. 18, No. 12 2019 “~Continuing Epoxy Resin CAS Number Story~ Curing Agent CAS Number Memorandum Part 31 Bismaleimide (1)” and MATERIAL STAGE Vol. 19, No. 2 2019 “~Continuing Epoxy Resin CAS Number Story~ Curing Agent CAS Number Memorandum Part 32 Bismaleimide (2)”, etc., but not limited to these. In addition, these may be used alone or in combination.

### [Cyanate Ester Resin]

The cyanate ester resin is a cyanate ester compound obtained by reacting phenolic resin with halogenated cyanide. Specific examples include dicyanatobenzene, tricyanatobenzene, dicyanatonaphthalene, dicyanatobiphenyl, 2,2′-bis(4-cyanatophenyl) propane, bis(4-cyanatophenyl) methane, bis(3,5-dimethyl-4-cyanatophenyl) methane, 2,2′-bis(3,5-dimethyl-4-cyanatophenyl) propane, 2,2′-bis(4-cyanatophenyl) ethane, 2,2′-bis(4-cyanatophenyl) hexafluoropropane, bis(4-cyanatophenyl) sulfone, bis(4-cyanatophenyl)thioether, phenol novolac cyanate, a compound obtained by converting the hydroxyl groups of phenol-dicyclopentadiene co-condensate to cyanate groups, etc., but not limited to these. In addition, these may be used alone or in combination.

Additionally, the cyanate ester compound whose synthesis method is described in Japanese Patent Application Laid-Open No. 2005-264154 is particularly preferable as the cyanate ester compound because the compound is excellent in low moisture absorption, flame retardancy, and dielectric properties.

The cyanate ester resin can also include catalysts such as zinc naphthenate, cobalt naphthenate, copper naphthenate, lead naphthenate, zinc octylate, tin octylate, lead acetylacetonate, and dibutyltin maleate to form sym-triazine rings by trimerizing the cyanate groups as required.

It is preferable to use the catalyst in an amount of 0.0001 parts by mass to 0.10 parts by mass, more preferably 0.00015 parts by mass to 0.0015 parts by mass, per 100 parts by mass of the cyanate ester resin.

### [Polybutadiene and Modified Products Thereof]

Polybutadiene and modified products thereof refer to polybutadiene or compounds having a structure derived from polybutadiene in the molecules. The structure derived from polybutadiene may have some or all of the unsaturated bonds converted to single bonds by hydrogenation.

Examples of the polybutadiene and modified products thereof include polybutadiene, hydroxyl-terminated polybutadiene, (meth)acrylate-terminated polybutadiene, carboxylic acid-terminated polybutadiene, amine-terminated polybutadiene, styrene-butadiene rubber, etc., but not limited to these. In addition, these may be used alone or in combination. Among these, polybutadiene or styrene-butadiene rubber is preferable from the viewpoint of dielectric properties. Examples of the styrene-butadiene rubber (SBR) include RICON-100, RICON-181, RICON-184 (all manufactured by Cray Valley), 1,2-SBS (manufactured by Nippon Soda), etc. Examples of the polybutadiene include B-1000, B-2000, and B-3000 (all manufactured by Nippon Soda). The preferable weight average molecular weight of polybutadiene and styrene-butadiene rubber is 500 to 10000, more preferably 750 to 7500, and even more preferably 1000 to 5000. When the weight average molecular weight is equal to or lower than the lower limit of the above range, the amount of volatilization increases, making it difficult to adjust the solid content during prepreg preparation; and when the weight average molecular weight is equal to or higher than the upper limit of the above range, the compatibility with other curable resins deteriorates. Generally, in the case of compounds containing heteroatoms such as oxygen or nitrogen, like bismaleimide or polymaleimide, it is difficult to ensure compatibility with low-polarity compounds such as compounds composed mainly of hydrocarbons or composed only of hydrocarbons, due to the polarity. On the other hand, by introducing alkenyl groups, the compound of the present invention has low polarity compared to maleimide compounds, resulting in excellent dielectric properties and low water absorption properties, and excellent compatibility with materials having low polarity and low dielectric properties, and compounds composed only of hydrocarbons.

### [Polystyrene and Modified Products Thereof]

Polystyrene and modified products thereof refer to polystyrene or compounds having a structure derived from polystyrene in the molecules.

Examples of the polystyrene and modified products thereof include polystyrene, styrene-2-isopropenyl-2-oxazoline copolymer (Epocros RPS-1005, RP-61, both manufactured by Nippon Shokubai), SEP (styrene-ethylene-propylene copolymer: Septon 1020 manufactured by Kuraray), SEPS (styrene-ethylene-propylene-styrene copolymer: Septon 2002, Septon 2004F, Septon 2005, Septon 2006, Septon 2063, Septon 2104, all manufactured by Kuraray), SEEPS (styrene-ethylene/ethylene-propylene-styrene block copolymer: Septon 4003, Septon 4044, Septon 4055, Septon 4077, Septon 4099, all manufactured by Kuraray), SEBS (styrene-ethylene-butylene-styrene block copolymer: Septon 8004, Septon 8006, Septon 8007L, all manufactured by Kuraray), SEEPS-OH (compound having a hydroxyl group at the terminal of styrene-ethylene/ethylene-propylene-styrene block copolymer: Septon HG252 manufactured by Kuraray), SIS (styrene-isoprene-styrene block copolymer: Septon 5125, Septon 5127, both manufactured by Kuraray), hydrogenated SIS (hydrogenated styrene-isoprene-styrene block copolymer: Hybrar 7125F, Hybrar 7311F, both manufactured by Kuraray), SIBS (styrene-isobutylene-styrene block copolymer: SIBSTAR073T, SIBSTAR102T, SIBSTAR103T (all manufactured by Kaneka), Septon V9827 (manufactured by Kuraray)), but not limited to these. In addition, these may be used alone or in combination. Polystyrene and modified products thereof have higher heat resistance and are less susceptible to oxidative degradation, and therefore preferably do not have unsaturated bonds. There is no particular limitation on the weight average molecular weight of polystyrene and modified products thereof as long as the weight average molecular weight is 10000 or more. However, when the weight average molecular weight is too large, the compatibility with polyphenylene ether compounds, low molecular weight components with a weight average molecular weight of about 50 to 1000, and oligomer components with a weight average molecular weight of about 1000 to 5000 deteriorates, making it difficult to ensure mixing and solvent stability. Therefore, the weight average molecular weight is preferable to be about 10000 to 300000.

### [Polyethylene and Modified Products Thereof]

Polyethylene and modified products thereof refer to polyethylene or compounds having a structure derived from polyethylene in the molecules. Examples of the polyethylene and modified products thereof include ethylene-propylene copolymer, ethylene-styrene copolymer, ethylene-propylene-ethylidene norbornene copolymer (EBT manufactured by Mitsui Chemicals: K-8370EM, K-9330M, etc.), ethylene-propylene-vinyl norbornene copolymer (VNB-EPT manufactured by Mitsui Chemicals: PX-006M, PX-008M, PX-009M, etc.), ethylene-vinyl alcohol copolymer, ethylene-vinyl acetate copolymer, etc., but not limited to these. From the viewpoint of improving heat resistance, it is preferable to use ethylene-propylene-ethylidene norbornene copolymer or ethylene-propylene-vinyl norbornene copolymer containing a crosslinkable structure. These may be used alone or in combination. There is no particular limitation on the weight average molecular weight of polyethylene and modified products thereof as long as the weight average molecular weight is 10000 or more. However, if the weight average molecular weight is too large, the compatibility with polyphenylene ether compounds, low molecular weight components with a weight average molecular weight of about 50 to 1000, and oligomer components with a weight average molecular weight of about 1000 to 5000 deteriorates, making it difficult to ensure mixing and solvent stability. Therefore, the weight average molecular weight is preferable to be about 10000 to 300000.

The curable resin composition of the present invention is obtained by preparing the above-mentioned components in predetermined proportions, and is sufficiently cured by pre-curing in the range of 130° C. to 180° C. for 30 seconds to 500 seconds, and further by post-curing at 150° C. to 200° C. for 2 hours to 15 hours, thereby obtaining the cured product of the present invention. In addition, the components of the curable resin composition can be uniformly dispersed or dissolved in a solvent or the like, and post-cured after removing the solvent.

The method for preparing the curable resin composition of the present invention is not particularly limited, and the curable resin composition may be prepared by simply mixing each component uniformly or by prepolymerization. For example, prepolymerization is performed by heating a mixture containing the compound of the present invention in the presence or absence of a curing accelerator or a polymerization initiator, and in the presence or absence of a solvent.

Similarly, prepolymerization may be performed by adding compounds such as amine compounds, compounds having ethylenic unsaturated bonds, maleimide compounds, cyanate ester compounds, polybutadiene and modified products thereof, polystyrene and modified products thereof, inorganic fillers, and other additives. Mixing of each component or prepolymerization is carried out using, for example, an extruder, a kneader, or rolls in the absence of a solvent, or using a reaction vessel equipped with a stirring device in the presence of a solvent.

As a method for uniform mixing, the components are mixed by kneading using devices such as a kneader, rolls, or a planetary mixer at a temperature in the range of 50° C. to 100° C. to obtain a uniform resin composition. The obtained resin composition can be ground and then molded into cylindrical tablets using a molding machine such as a tablet machine, or formed into granular powder or powdery molded bodies, or these compositions can be melted on a surface support and molded into a sheet with a thickness of 0.05 mm to 10 mm, to form a curable resin composition molded body. The obtained molded body becomes a non-sticky molded body at 0° C. to 20° C., and does not significantly decrease in fluidity and curability even when stored for one week or longer at −25° C. to 0° C.

The obtained molded body can be molded into a cured product using a transfer molding machine or a compression molding machine.

The curable resin composition of the present invention can also be made into a varnish-like composition (hereinafter simply referred to as varnish) by adding an organic solvent. The curable resin composition of the present invention can be dissolved in a solvent such as toluene, xylene, acetone, methyl ethyl ketone, methyl ketone, dimethylformamide, dimethylacetamide, N-methylpyrrolidone, etc., as required, to form a varnish. The varnish can be impregnated into a substrate such as glass fibers, carbon fibers, polyester fibers, polyamide fibers, alumina fibers, paper, etc., and heat-dried to obtain a prepreg. The cured product of the curable resin composition of the present invention can be obtained by thermal press molding of the obtained prepreg. In this case, the solvent is used in an amount occupying 10 weight % to 70 weight %, preferably 15 weight % to 70 weight %, in the mixture of the curable resin composition of the present invention and the solvent. In addition, if the curable resin composition is a liquid composition, a curable resin composition containing carbon fibers can be obtained directly, for example, by a resin transfer molding (RTM) method.

Moreover, the curable resin composition of the present invention can also be used as a modifier for a film-type composition. Specifically, the curable resin composition can be used to improve flexibility, etc. in the B-stage. Such a film-type resin composition is obtained as a sheet-like adhesive by applying the curable resin composition of the present invention as the aforementioned curable resin composition varnish on a release film, removing the solvent under heating, and then B-staging. This sheet-like adhesive can be used as an interlayer insulation layer in a multilayer substrate or the like.

The curable resin composition of the present invention can also be used to obtain a prepreg by heating and melting, reducing viscosity, and impregnating reinforcing fibers such as glass fibers, carbon fibers, polyester fibers, polyamide fibers, alumina fibers, etc. Specific examples include glass fibers such as E-glass cloth, D-glass cloth, S-glass cloth, Q-glass cloth, spherical glass cloth, NE-glass cloth, and T-glass cloth, as well as inorganic fibers other than glass and organic fibers such as poly-p-phenylene terephthalamide (Kevlar (registered trademark), manufactured by Dupont), all-aromatic polyamide, polyester, poly-p-phenylene benzoxazole, polyimide, and carbon fibers, but not particularly limited to these. The shape of the substrate is not particularly limited, but examples include woven fabric, non-woven fabric, roving, chopped strand mat, etc. As for the weaving method for woven fabric, plain weave, basket weave, twill weave, etc. are known, and these known methods can be appropriately selected and used according to the intended application and performance. In addition, spread woven fabric or glass woven fabric surface-treated with a silane coupling agent or the like is suitably used. The thickness of the substrate is not particularly limited, but is preferably about 0.01 mm to 0.4 mm. Furthermore, a prepreg can also be obtained by impregnating the aforementioned varnish into reinforcing fibers and heat-drying.

Additionally, a laminate can also be manufactured using the aforementioned prepreg. The laminate is not particularly limited as long as the laminate includes one or more sheets of prepreg, and may have any other layers. The manufacturing method for the laminate can appropriately apply generally known methods and is not particularly limited. For example, in the case of forming a metal foil-clad laminate, multi-stage press machines, multi-stage vacuum press machines, continuous molding machines, autoclave molding machines, etc. can be used, and the laminate can be obtained by stacking the aforementioned prepregs and heat pressure molding. In this case, the heating temperature is not particularly limited, but 65° C. to 300° C. is preferable, and 120° C. to 270° C. is more preferable. Moreover, the pressure for pressing is not particularly limited, but when the pressure is too high, it becomes difficult to adjust the solid content of the resin in the laminate and the quality becomes unstable, and when the pressure is too low, bubbles and interlayer adhesion deteriorate, so 2.0 MPa to 5.0 MPa is preferable, and 2.5 MPa to 4.0 MPa is more preferable. The laminate of this embodiment can be suitably used as a metal foil-clad laminate, which will be described later, by including a layer composed of metal foil.

An electrical electronic laminate (printed circuit board) or carbon fiber reinforced material can be obtained by cutting the above prepreg into a desired shape, laminating the prepreg with copper foil or the like as required, and then heat-curing the curable resin composition while applying pressure to the laminate using methods such as press molding, autoclave molding, sheet winding molding or the like.

The curable resin composition of the present invention can also be made into a resin sheet. As a method for obtaining a resin sheet from the curable resin composition of the present invention, for example, the curable resin composition is applied on a support film (support) and then dried to form a resin composition layer on the support film. In the case of using the curable resin composition of the present invention for a resin sheet, it is essential that the film softens at the lamination temperature conditions (70° C. to 140° C.) in a vacuum lamination method, and shows flowability (resin flow) that enables resin filling in via holes or through holes existing in the circuit board simultaneously with the lamination of the circuit board. It is preferable to blend the aforementioned components to exhibit such characteristics. It should be noted that, in the obtained resin sheet and circuit board (such as copper-clad laminate), it is required to have appearance uniformity in order to prevent phenomena such as showing locally different characteristic values due to phase separation, and to exhibit constant performance at any part.

Here, the diameter of the through holes in the circuit board is 0.1 mm to 0.5 mm, and the depth is 0.1 mm to 1.2 mm, and it is preferable to enable resin filling within this range. In the case of laminating on both sides of the circuit board, it is desirable that the through holes are filled to about ½.

As a specific method for manufacturing the aforementioned resin sheet, for example, a varnished resin composition is prepared by blending an organic solvent, then the varnished resin composition is applied to the surface of a support film (Y), and further, the organic solvent is dried by heating or blowing hot air, to form a resin composition layer (X).

As the organic solvent used here, it is preferable to use, for example, ketones such as acetone, methyl ethyl ketone, cyclohexanone; acetate esters such as ethyl acetate, butyl acetate, cellosolve acetate, propylene glycol monomethyl ether acetate, carbitol acetate; carbitols such as cellosolve, butyl carbitol; aromatic hydrocarbons such as toluene, xylene; dimethylformamide, dimethylacetamide, N-methylpyrrolidone, etc. It is also preferable to use these in a proportion that results in 30 mass % to 60 mass % of non-volatile content.

The thickness of the aforementioned resin composition layer (X) formed needs to be equal to or greater than the thickness of the conductor layer. Since the thickness of the conductor layer included in the circuit board is in the range of 5 μm to 70 μm, it is preferable that the thickness of the resin composition layer (X) is 10 μm to 100 μm. The resin composition layer (X) in the present invention may be protected by a protective film which will be described later. With protection of the protective film, it is possible to prevent the adhesion of dust and scratches on the surface of the resin composition layer.

Examples of the aforementioned support film and protective film can include polyolefins such as polyethylene, polypropylene, polyvinyl chloride; polyesters such as polyethylene terephthalate (PET), polyethylene naphthalate; polycarbonate, polyimide, and further, release paper or metal foils such as copper foil and aluminum foil. The support film and the protective film may be subjected to matte treatment, corona treatment, or release treatment. The thickness of the support film is not particularly limited, but is 10 μm to 150 μm, and is preferably used in the range of 25 μm to 50 μm. Additionally, it is preferable that the thickness of the protective film is 1 μm to 40 μm.

The aforementioned support film (Y) is peeled off after laminating on the circuit board, or after forming an insulation layer by heat curing. If the support film (Y) is peeled off after the resin composition layer constituting the resin sheet has been heat-cured, it is possible to prevent the adhesion of dust during the curing process. In the case of peeling after curing, the support film is subjected to release treatment in advance.

A multilayer printed circuit board can be manufactured from the resin sheet obtained as described above. For example, in the case where the aforementioned resin composition layer (X) is protected by the protective film, after peeling these off, the layer (X) of the resin composition is laminated on one side or both sides of the circuit board so as to directly contact the circuit board, for example, by a vacuum lamination method. The lamination method may be batch-type or continuous-type using rolls. Furthermore, if necessary, the resin sheet and the circuit board may be heated (preheated) as required before lamination. As for the lamination conditions, the press-bonding temperature (lamination temperature) is preferably 70° C. to 140° C., the press-bonding pressure is preferably 1 kgf/cm² to 11 kgf/cm² (9.8×10⁴ N/m² to 107.9×10⁴ N/m²), and it is preferable to laminate under reduced pressure with an air pressure of 20 mmHg (26.7 hPa) or less.

In addition, a semiconductor device can be manufactured using the curable resin composition of the present invention. Examples of the semiconductor device include DIP (Dual In-line Package), QFP (Quad Flat Package), BGA (Ball Grid Array), CSP (Chip Size Package), SOP (Small Outline Package), TSOP (Thin Small Outline Package), TQFP (Thin Quad Flat Package), etc.

The curable resin composition of the present invention and the cured product thereof can be used in a wide range of fields. Specifically, the curable resin composition of the present invention and the cured product thereof can be used for various applications such as molding materials, adhesives, composite materials, and paints. Since the cured product of the curable resin composition described in the present invention exhibits excellent heat resistance and dielectric properties, the cured product is suitably used for electrical and electronic components such as encapsulants for semiconductor elements, encapsulants for liquid crystal display elements, encapsulants for organic EL elements, laminates (printed circuit boards, substrates for BGA, build-up substrates, etc.), composite materials for lightweight high-strength structural materials such as carbon fiber reinforced plastics and glass fiber reinforced plastics, 3D printing, etc.

### EXAMPLES

The present invention will now be described more specifically based on examples. Hereinafter, unless otherwise specified, “parts” refers to parts by mass. It should be noted that the present invention is not limited to these examples.

The following describes various analytical methods used in the examples.

### <Gel Permeation Chromatography (GPC)>

The weight average molecular weight (Mw) and the number average molecular weight (Mn) were calculated by polystyrene conversion using polystyrene standard solution.
GPC: DGU-20A3R, LC-20AD, SIL-20AHT, RID-20A, SPD-20A, CTO-20A, CBM-20A (all manufactured by Shimadzu Corporation)
Column: Shodex KF-603, KF-602×2, KF-601× 2)
Linked eluent: Tetrahydrofuran
Flow rate: 0.5 ml/min.
Column temperature: 40° C.
Detection: RI (differential refractive index detector)

### Example 1

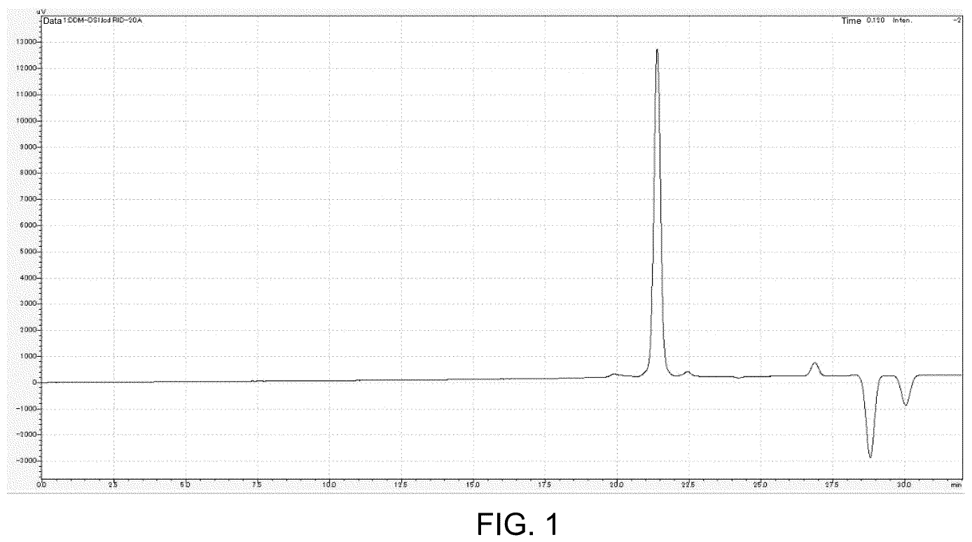

While applying nitrogen purge to a flask equipped with a thermometer, a cooling tube, a fractionation tube, and a stirrer, 42.1 parts of octenyl succinic anhydride, 116 parts of toluene, and 19.8 parts of 4,4′-diaminodiphenylmethane were added, and stirring was initiated. The internal temperature was raised to 112° C. over 2 hours, and the reaction was carried out for 2 hours. 1.0 part of methanesulfonic acid was added, and the reaction was carried out at 113° C. to 115° C. for 2 hours while removing the generated water from the system. After cooling, the organic layer was washed with water until the drainage became neutral until the wastewater became neutral, and then concentrated to obtain 57.4 parts of compound (A1) represented by the following formula (1-a) as a brown liquid resin. The GPC chart of the obtained compound (A1) is shown in FIG. 1.

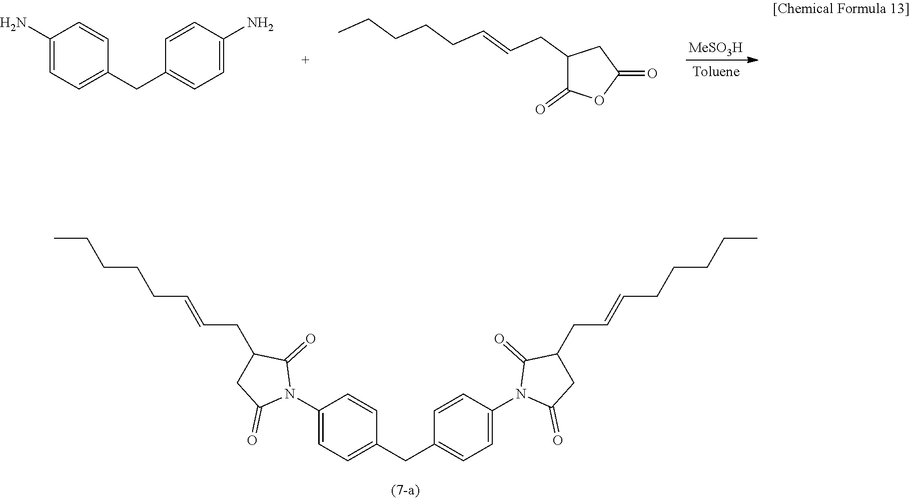 [SMILES: "MeSO3H" ; Nc1ccc(Cc2ccc(N)cc2)cc1 ; CCCCC/C=C/CC1CC(=O)OC1=O ; CCCCC/C=C/CC1CC(=O)N(c2ccc(Cc3ccc(N4C(=O)CC(C/C=C/CCCCC)C4=O)cc3)cc2)C1=O]

### Example 2

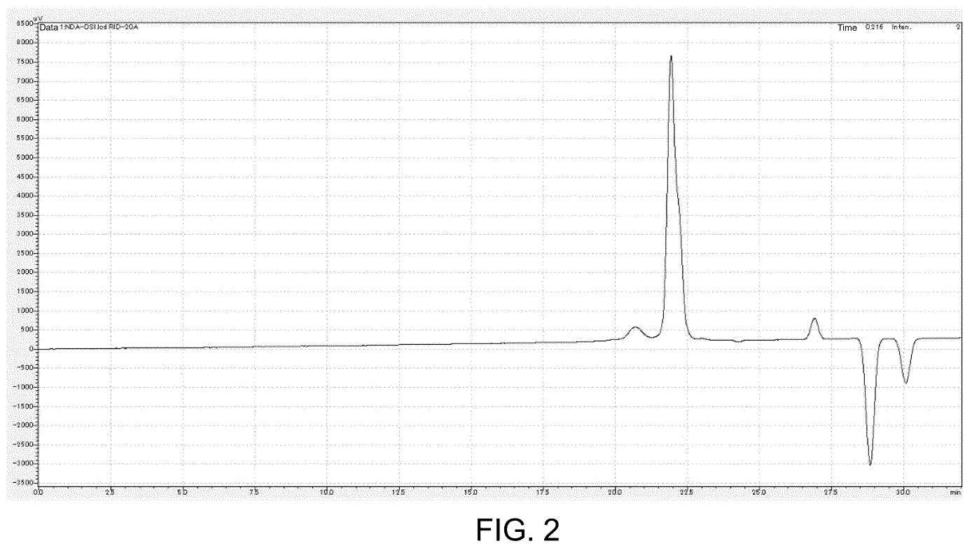

While applying nitrogen purge to a flask equipped with a thermometer, a cooling tube, a fractionation tube, and a stirrer, 42.0 parts of octenyl succinic anhydride, 100 parts of xylene, 15 parts of triethylamine, and 15.4 parts of norbornane diamine (manufactured by Mitsui Chemicals Fine Inc.) were added, and stirring was initiated. The mixture was added dropwise at an internal temperature of 120° C. or lower, and the reaction was carried out at 120° C. for 1 hour, at 130° C. for 2 hours, and at 150° C. for 9 hours. The obtained reaction solution was concentrated to obtain 49.9 parts of compound (A2) represented by the following formula (2-a) as a brown liquid resin. The GPC chart of the obtained compound (A2) is shown in FIG. 2.

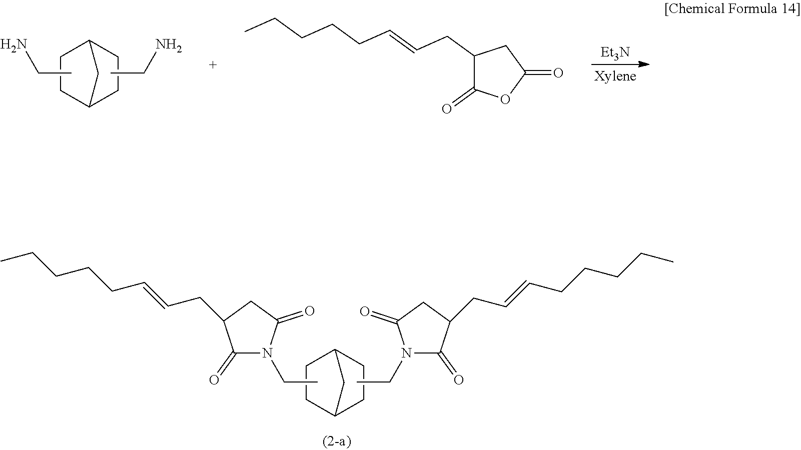

### Examples 3, 4, Comparative Examples 1, 2

The compounds obtained in Examples 1 and 2, and the materials listed in Table 1 were blended in the proportions (parts by mass) shown in Table 1, sandwiched between mirror-finished copper foils (T4X: manufactured by Fukuda Metal Foil & Powder Co., Ltd.), vacuum press molded, and cured at 220° C. for 2 hours. At this time, a cushion paper with a thickness of 250 μm and a 150 mm square cut out from the center was used as a spacer. During evaluation, test pieces were cut to the desired size using a laser cutter as required, and the evaluation was carried out. The evaluation results are shown in Table 1.

### <Heat Resistance (DMA)>

Dynamic viscoelasticity measuring instrument: TA-instruments, DMA-2980
Measurement temperature range: −30° C. to 280° C.
Temperature increase rate: 2° C./min
Frequency: 10 Hz
Measurement mode: Tension
Test piece size: A piece cut to 5 mm×50 mm was used (thickness was 0.2 mm)
Tg: The peak point of tan 8 (=loss modulus/storage modulus) was defined as Tg

### <Dielectric Constant Test and Dielectric Loss Tangent Test>

The test was carried out using a 10 GHz cavity resonator manufactured by ATE Co., Ltd., at 25° C. by the cavity resonator perturbation method. The sample size was set to 1.7 mm in width×100 mm in length, and the test was carried out with a thickness of 0.3 mm.

TABLE 1

| | | | | Compar- | Compar- |
|---|---|---|---|---|---|
| | | | | ative | ative |
| | | Example 3 | Example 4 | Example 1 | Example 2 |
| Maleimide | MI | 51.0 | 52.5 | 100 | 50 |
| compound | | | | | |
| Compound | A1 | 49.0 | | | |
| | A2 | | 47.5 | | |
| Polyphenylene | SA- | | | | 50 |
| ether | 9000 | | | | |
| compound | | | | | |
| Polymerization | DCP | 1 | 1 | 1 | 1 |
| initiator | | | | | |
| Imidazole | 2E- | | | | |
| | 4MZ | | | | |

Evaluation result

| | | | | | |
|---|---|---|---|---|---|
| Tg | ° C. | 209 | 209 | 350 or | 214 |
| | | | | more | |

| | | | | |
|---|---|---|---|---|
| Dielectric constant | 2.7 | 2.6 | 2.7 | 2.7 |
| (10 GHz) | | | | |
| Dielectric loss | 0.0023 | 0.0023 | 0.0034 | 0.0029 |
| tangent (10 GHz) | | | | |

MI: A compound solidified by removing the solvent under reduced pressure from the resin solution containing the maleimide compound (M2) described in Example 4 of Japanese Patent Application Laid-Open No. 2009-001783
SA-9000: Terminal methacrylated polyphenylene ether compound (manufactured by SABIC)
DCP: Dicumyl peroxide (manufactured by Kayaku Akzo)
2E4MZ: 2-Ethyl-4-methylimidazole (curing accelerator, manufactured by Shikoku Chemical)

It was confirmed from the results of Comparative Example 1 that, in the case where the maleimide compound was subjected to radical polymerization alone (Comparative Example 1), the heat resistance was good, but the dielectric loss tangent was poor. In contrast, Examples 3 and 4 which included the compound of the present invention showed a 32.4% improvement in dielectric loss tangent compared to Comparative Example 1. On the other hand, Comparative Example 2 which included the polyphenylene ether compound exhibited heat resistance equivalent to Examples 3 and 4, but the improvement in dielectric loss tangent was limited to 14.7% compared to Comparative Example 1.

### Examples 5, 6, Comparative Example 3

### <Solvent Solubility Test>

In a 5 mL glass sample vial, 1.0 g each of A1, A2, and phenylmethane maleimide (abbreviated as PMMI, CAS No.: 67784-74-1) were combined with 1.0 g each of toluene. The mixtures were then stirred for 1 hour at 25° C. using a roller mixer (MIX-ROTAR VMR-5: manufactured by ASONE) at a rotation speed of 100 rpm to prepare a toluene solution containing 50 wt % solids of each compound. The obtained solution was visually observed and evaluated according to the following criteria. The results are shown in Table 2.
Case where a uniform solution was obtained: O
Case where a uniform solution was not obtained: x

TABLE 2

| | | | | Comparative |
|---|---|---|---|---|
| | | Example 5 | Example 6 | Example 3 |
| Compound | A1 | 50 | | |
| | A2 | | 50 | |
| Maleimide compound | PMMI | | | 50 |
| Solvent | Toluene | 50 | 50 | 50 |

Evaluation result

| | | | |
|---|---|---|---|
| Solvent solubility | ∘ | ∘ | x |

From the results in Table 2, it was confirmed that the compound of the present invention can exhibit solvent solubility that cannot be achieved with maleimide groups by introducing alkenyl groups and succinimide structures into the molecule, thereby reducing intermolecular interactions and lowering crystallinity.

### INDUSTRIAL APPLICABILITY

The curable resin composition, the resin sheet, and the cured product thereof according to the present invention are suitably used for electrical and electronic components such as semiconductor encapsulants, printed circuit boards, and build-up laminates, as well as lightweight high-strength materials such as carbon fiber reinforced plastics and glass fiber reinforced plastics, and for 3D printing applications.

To the claims:

1. A compound obtained by reacting a compound represented by following formula (a) with a compound having two or more amino groups in a molecule, 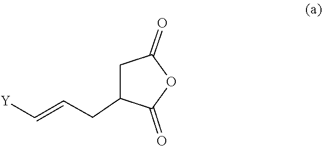
(in above formula (a), Y represents a hydrocarbon group having 1 to 20 carbon atoms).

2. The compound according to claim 1, wherein the compound having two or more amino groups in the molecule comprises an alicyclic ring or an aromatic ring.

3. The compound according to claim 1, represented by following formula (b), 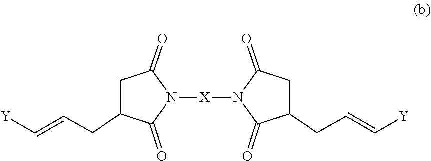
(in above formula (b), a plurality of Y independently represent hydrocarbon groups having 1 to 20 carbon atoms, and X represents one or more of following formulas (A) to (T)), 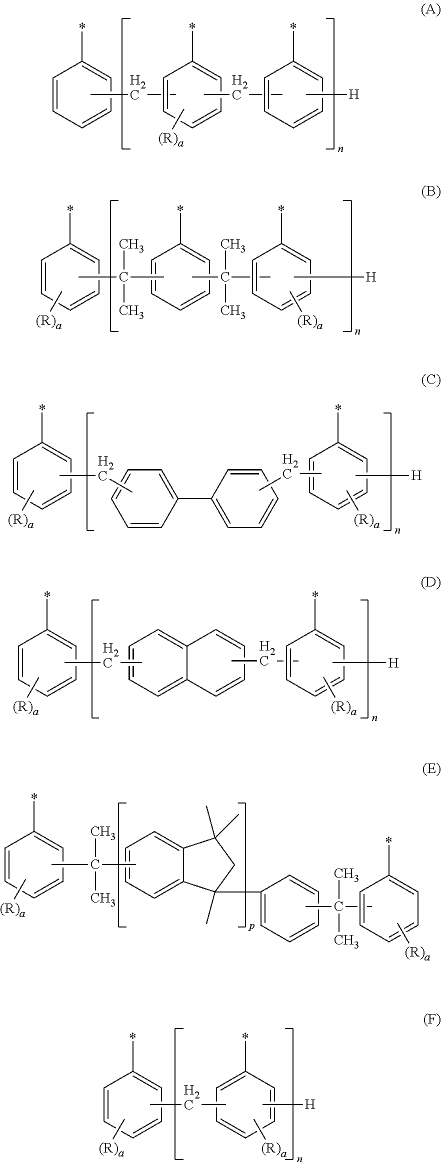 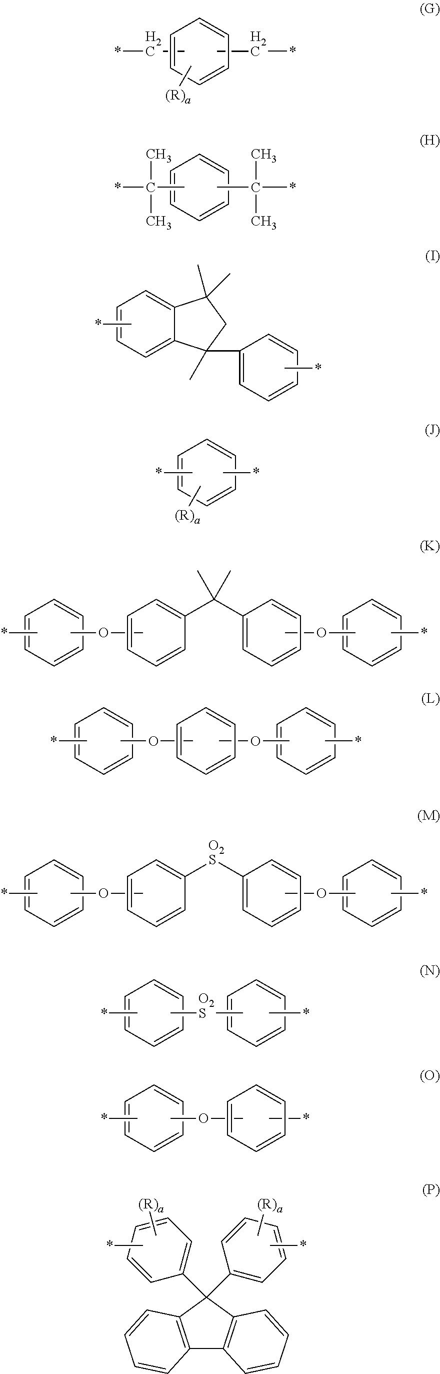 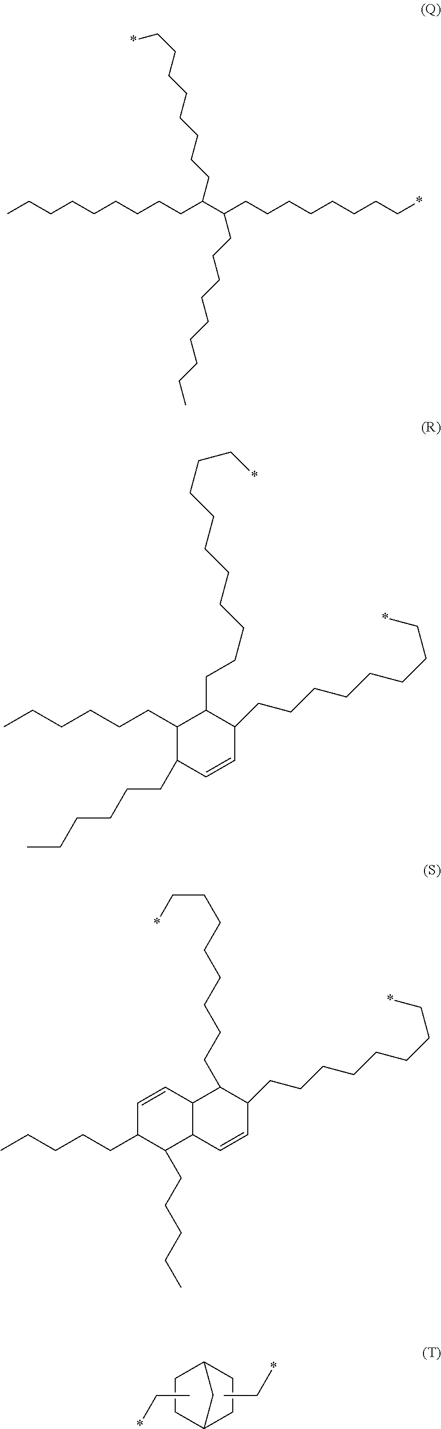
(in above formulas (A) to (T), R represents a hydrogen atom or a hydrocarbon group having 1 to 10 carbon atoms, a represents an integer of 1 to 4, p and n are each the number of repetitions and represent a number of 1 to 20, and * indicates a bonding position with a nitrogen atom in formula (2)).

4. A curable resin composition, comprising the compound according to claim 1.

5. The curable resin composition according to claim 4, further comprising a radical polymerization initiator.

6. The curable resin composition according to claim 4, further comprising a maleimide compound.

7. A cured product, obtained by curing the curable resin composition according to claim 4.

8. A cured product, obtained by curing the curable resin composition according to claim 5.

9. A cured product, obtained by curing the curable resin composition according to claim 6.
